(19) United States
(12) Patent Application Publication
(10) Pub. No.: US 2025/0157573 A1
(43) Pub. Date: May 15, 2025

(54) GENOME WIDE ASSEMBLY-BASED STRUCTURAL VARIANT CALLING

(71) Applicant: Illumina, Inc., San Diego, CA (US)

(72) Inventors: Camilla Colombo, Cambridge (GB); Mitchell A. Bekritsky, Decatur, GA (US); Ritu Kundu, Caldecote (GB); Stefan Dorokhov, Birmingham (GB)

(73) Assignee: Illumina, Inc., San Diego, CA (US)

(21) Appl. No.: 18/887,480

(22) Filed: Sep. 17, 2024

Related U.S. Application Data
(60) Provisional application No. 63/597,521, filed on Nov. 9, 2023.

(51) Int. Cl. G16B 20/20; G16B 20/10; G16B 30/10

(52) U.S. Cl. CPC G16B 20/20; G16B 20/10; G16B 30/10

(57) ABSTRACT

This disclosure describes systems, non-transitory computer readable media, and methods for implementation of SV calling pipelines that can generate high-confidence diploid assemblies and SV call sets based on trio read data. For instance, a system is provided for implementing an SV calling pipeline via a bioinformatics subsystem executed on one or more processors that can receive a trio dataset of diploid read data of sequenced child (CH) and parental (M, P) genomes, and generate, based on consensus regions of the trio read data, a validated diploid assembly of the CH genome. Validation can be determined based on composition vectors of corresponding child and transmitting parent sequences at a given interval for each consensus region. The bioinformatics subsystem can then detect structural variants (SV) within the CH diploid assembly and output a VCF file of detected SVs as an SV truthset.

### CROSS-REFERENCE TO RELATED APPLICATIONS

This application claims priority to U.S. Provisional Application No. 63/597,521, filed Nov. 9, 2023, the entire disclosure of which is hereby incorporated by reference herein in its entirety.

### BACKGROUND

Next Generation Sequencing (NGS) and variant calling algorithms developed to discover various forms of pathologic genomic variants are commonly used by clinical laboratories to determine the molecular etiology of complex diseases or disorders and in many cases to make critical treatment course decisions. Structural variants (or SVs), in particular, are a major source of human genetic diversity—accounting for more nucleotide changes than any other variant class—and have been linked to numerous diseases and other phenotypes of interest. Thus they are a key focus of modern pharmacogenetics.

Due to their large size, complexity, and diversity, SVs are exceptionally difficult to accurately characterize and detect, and require, paradoxically, simultaneously large-scale, yet highly-refined genomic analysis. For example, SV calling strategies are commonly based on haplotype-resolved reconstruction of whole genome (or exome) sequencing data, where analysis is largely performed based on computational methods at each stage of generating, aligning, interpreting, and validating such data. The accuracy and reproducibility of SV detection or discovery in a clinical setting thus depends on multiple factors, including reproducible coverage bias of the sequencing technique and the performance of software tools for read-alignment, variant-calling, and other pipelined processes.

Software tools for SV calling are heavily reliant on genomic reference data as consensus models, which may be variously used as: scaffolding for read-alignment; SV truthsets for variant detection; and gold standards for benchmarking. The ideal genomic reference resource from which to draw for these purposes would comprise a single contiguous sequence (contig) of each chromosome, where the order and orientation of the complete chromosome sequence are resolved from telomere to telomere, and have pan-genomic allelic representation and variant-awareness.

The human reference genome (GRCh38) has been the gold standard for mammalian genomes since its first publication in 2001, and there has been considerable investment over the past two decades to increase its accuracy and contiguity. Notwithstanding, even in its current iteration, the number of contiguous sequences (contigs) greatly exceeds the number of chromosomes (1006 contigs versus 24 chromosomes), with most of the major gaps corresponding to large repetitive sequences present in centromeres, acrocentric DNA and segmental duplications. Moreover, approximately 93% of the primary assembly (the assembled chromosomes, unlocalized and unplaced sequences) consists of sequences from a mere 11 genomic clone libraries, and includes only a limited number of alternative contigs spanning only 60 Mb of the total 2.9 Gb ungapped length of the primary assembly.

As a consequence of the lack of diversity, sample size and depth of GRCh38 and other reference sets, comparative progress in SV analysis in relation to other areas of genomic study is lagging.

### SUMMARY

Automated systems for implementation of SV calling pipelines described herein can generate high-confidence diploid assemblies and SV call sets from trio read data through automation and internalization of the validation process. For example, pipeline methods described herein analyze composition vectors of trio input data to cycle back information for use in validating diploid assembly and SV call outputs. Implementing such methods, SV calling pipelines can perform turn-key operations for trio reads and can analyze trio read data with a high degree of accuracy and over a wide range of genetic diversity. Thus, in one aspect, systems herein can implement automated trio-based assembly of high-quality diploid human reference genomes, benchmark SV sets, and pan-genomic graphs. Similarly, in another aspect, systems can refine, diversify and/or expand existing reference constructs and benchmark SV sets through automated compiling of genomic data from multiple sampled trios.

In particular embodiments, a system is provided for implementing an SV calling pipeline via a bioinformatics subsystem executed on one or more processors. The bioinformatics subsystem can receive a trio dataset of diploid read data of sequenced child (CH) and parental (M, P) genomes, and generate, based on the read data, aligned consensus regions in haplotype-specific (phased) child and parental sets, i.e., sets of CHH1/CHH2-MH1/MH2-PH1/PH2 consensus regions (three diploid sets/six haplotype sequence sets). The bioinformatics subsystem can validate the consensus regions of the child diploid set and output, in an alignment format, a datafile (e.g., a BED file) of validated child consensus regions as a diploid assembly. In one example, validation for each consensus region is determined based on composition vectors of corresponding child and transmitting parent sequences at a given interval, e.g., interval sequences of M-CHH1 and either of MH1/MH2 and P-CHH2 and either of PH1/HP2. The bioinformatics subsystem can then analyze the datafile and detect structural variants (SV) within the stored diploid assembly and output, in a variant call format, a datafile (e.g., a VCF file) of detected SVs as an SV call set.

In one example embodiment, a bioinformatics subsystem can include one or more secondary analysis subsystems for implementing an SV calling pipeline. For instance, the bioinformatics subsystem can include a first assembly subsystem, which can prepare, from a trio dataset, one or more alignment files of colinear and orthogonal alignments of phased CHH1/CHH2-MH1/MH2-PH1/PH2 contigs to a stored reference. In examples, the first assembly subsystem can first partition the trio read data set into phased CHH1/CHH2-MH1/MH2-PH1/PH2 read sets. The first assembly subsystem can next assemble the phased read sets into respective trio sets of contigs and then access a reference genome to generate alignment data of phased sets of CHH1/CHH2-MH1/MH2-PH1/PH2 contigs. For example, the first assembly subsystem can use a de novo assembly methodology using, e.g., a de bruijn or string graph.

The bioinformatics subsystem can also include a second assembly subsystem, which can analyze alignment data and generate phased sets CHH1/CHH2 consensus regions based on orthogonal (allelic) alignment with parental contigs. To that end, the second assembly subsystem can join adjacent contigs of each colinearly aligned haplotype sequence set into region assemblies according to one or more heuristics, in which, e.g., each of the one or more heuristic is a predicate expression of alignment between two adjacent contigs. The second assembly subsystem can then generate a consensus set among orthogonally aligned region assemblies (or allelic groupings) by trimming portions of either haplotype sequence of a given CHH1/CHH2 region that does not align with corresponding portions each haplotype sequence of respective parental regions of the allelic grouping. The second assembly subsystem can then compress consensus data to an alignment file for output as a phased diploid set of CHH1/CHH2 consensus regions.

In the same or yet another example, the validation subsystem can receive an alignment file storing a phased set of CHH1/CHH2 consensus regions and generate, based on composition vector analysis, an alignment file storing a validated set of CHH1/CHH2 consensus regions. For instance, the validation subsystem can determine composition vectors of each consensus region at intervals (I) based on a summing of k-mer frequency vectors, where the value k can be 1≤k≤(I) and a frequency vector (v) is determined for each (I)−k+1 number (n) of k-strings in a given interval (I) to yield v₁₋ₙ k-mer frequency vectors per interval (I). The validation subsystem can then determine the validity of each child consensus region based on the determined composition vector for each haplotype. In examples, a child consensus region at a given interval (I) can be determined valid if the composition vector of each sequence of respective haplotypes is within a threshold distance measure of the composition vector of one of the two sequences of respective haplotypes of a transmitting parent (i.e., between M-CHH1-MH1/MH2 and P-CHH2-PH1/PH2 sequences). In one particular example, the child consensus region at a given interval (I) can be determined valid if each sequence of respective haplotypes of the child and one of the two sequences of respective haplotypes of a transmitting parent meets a cosine similarity (cos(θ)) threshold of 90≤cos(θ)<100. For instance, in one example, the threshold cosine similarity value threshold can be in the range of ˜0.94 to ˜0.96.

CHH1/CHH2 sequences at a given interval (I) determined to be valid can be retained as child consensus regions and output, in an alignment format, as a diploid assembly; whereas sequences determined to be invalid can be discarded. Alternatively, sequences determined to be invalid may be subject to one or more additional validation procedures before any such sequences (or subsequences) are discarded. For instance, validation can be determined in two or more passes. In one example, each child consensus region can be validated, in a first pass, at an interval (I), and child sequences of interval (I) determined to be invalid in the first pass can be validated, in a second pass, at a subinterval (Iₛ). Any sequences at subinterval (Iₛ) not deemed valid can either be discarded or subject to a third pass, and so on.

In another aspect, the bioinformatics subsystem can include an SV caller subsystem for SV calling within a diploid assembly, including a diploid assembly generated according to one or more of the foregoing embodiments. In one example embodiment, the SV caller subsystem implements an SV calling protocol through operation of an SV caller engine and a call set engine. For instance, the SV caller engine can implement an SV caller to perform SV calling in a diploid assembly. Certain example embodiments perform non-reference based SV discovery, including SV discovery in repetitive and low complexity regions (RLCR) or noisy DNA loci of a diploid genome. The call set engine can phase, interpret, and annotate the variant calls generated by the SV caller engine to generate a high-confidence genome-wide variant call set.

According to some embodiments, the SV calling protocol can implement multiple-caller processes. In examples, the SV caller subsystem can operate a secondary SV caller adapted to analyze tandem repeat (TR) structures. In one example, validated CHH1/CHH2 consensus regions can be sorted into tandem repeat (TR) and non-tandem repeat (NTR) sets, and a secondary caller operates to identify copy number variation (CNV) and other SVs within the TR data. In the same or other examples, the SV calling protocol can implement combined molecular strategies. For instance, the SV caller engine can execute a multi-sample SV calling protocol incorporating various combinations of long-read analysis, paired-end read analysis, split-read analysis, linked read analysis, local reassembly (read-based) analysis.

Methods are also provided for generating diploid assemblies and variant call sets for tertiary analysis, including, e.g., comprehensive genomic profiling, Genome-wide Association Studies (GWAS), Variant to Function (V2F). QTL mapping, missense studies, loss of function/gain of function, conservation, depletion, deletion analyses. Methods further include generating VCF files with SV call sets and mutation, gene, and transcript annotation, and TSV files with gene expression models.

While this disclosure has been described in terms of certain embodiments and generally associated methods, alterations and permutations of the embodiments and methods will be apparent to those skilled in the art. Accordingly, the above description of example embodiments does not constrain this disclosure. Other changes, substitutions, and alterations are also possible without departing from the spirit and scope of this disclosure.

### BRIEF DESCRIPTION OF THE DRAWINGS

FIG. 1 is an illustration of six cannon structural variants.

FIG. 2 illustrates a schematic diagram of a system environment implementing a bioinformatics subsystem.

FIG. 3A shows an example of one or more secondary analysis subsystems that may be implemented as a diploid assembly and structural variant calling platform by a bioinformatics subsystem.

FIG. 3B shows an example illustration of a de Bruijn graph that may be implemented in an assembly engine of a diploid assembly and structural variant calling platform by a bioinformatics subsystem.

FIG. 3C shows mathematical and graphical representations cosine distance/similarity.

FIG. 3D shows a score histogram and table of results of threshold testing on sequence length loss and scatter plot of score versus interval length.

FIG. 3E shows a graph of results of threshold testing on sequence length loss.

FIG. 3F shows tabular results of two-pass validation testing.

FIG. 3G is a flow diagram of a process of an example SV caller that may be implemented in a diploid assembly and structural variant calling platform by a bioinformatics subsystem.

FIG. 3H shows an example secondary analysis subsystem that may be implemented as part of a diploid assembly and structural variant calling platform by a bioinformatics subsystem.

FIG. 4A is a flow diagram of an example procedure for generating a CHH1/CHH2 diploid assembly and SV call set from CHH1/CHH2-MH1/MH2-PH1/PH2 read sets.

FIG. 4B is a flow diagram of an example procedure for generating a CHH1/CHH2 diploid assembly and SV call set from CHH1/CHH2-MH1/MH2-PH1/PH2 read sets.

FIG. 5A shows an example of one or more methods that may be implemented on a diploid assembly and structural variant calling platform by a bioinformatics subsystem.

FIG. 5B shows an example of one or more methods that may be implemented on a diploid assembly and structural variant calling platform by a bioinformatics subsystem.

FIG. 5C shows an example of one or more methods that may be implemented on a diploid assembly and structural variant calling platform by a bioinformatics subsystem.

FIG. 5D shows example entries for VCF file headings for CNV calling in TR consensus regions.

FIG. 5E shows example filters applied to SV calls emitted to lines of a VCF.

FIG. 5F shows an example VCF file for a VC truthset output from an example method that may be implemented on a diploid assembly and structural variant calling platform by a bioinformatics subsystem.

FIG. 6 shows a diagram of a computer device.

### DETAILED DESCRIPTION

The following paragraphs describe the systems and method in the context of illustrative figures that portray example embodiments and implementations.

Importantly, the faithful reconstruction of a fully haplotype resolved genome from the millions of reads obtained during sequencing, as a computational problem, may generally be reduced to a series of smaller problems solved (never entirely, but to acceptable degree) using a pipelined ensemble of computational processes and molecular techniques, which may vary depending, e.g., on the choice of sequencing strategy and the approach and objectives to reconstruction. For example, there are two main approaches to reconstruction, one reference-based (e.g., read alignment) and the other nonreference-based (e.g., de novo assembly). A reference-based approach reduces the reconstruction problem, in part, to one of locating the original position of each read in the sequenced genome. For example, read alignment queries each read to a matching target of known position in a reference genome. The original position of the read in the sequence gene is assumed to be collocated at the position of the matching target the reference. By contrast, a nonreference-based approach assumes no apriori knowledge of the sequenced genome and, rather than locate the original position of the reads, the approach instead attempts to assemble the reads in a graph (e.g., de bruijn) based, in part, on observations of read overlap or partial alignment.

SV discovery, as an additional computational problem, may also be resolved using various or multiple approaches. SVs are extremely diverse in type and size, ranging anywhere from ˜50 bp to well over megabases of sequence, affecting more of the genome per nucleotide changes than any other class of sequence variant. SVs comprise myriad subclasses that consist of unbalanced copy number variants (CNVs), which include deletions, duplications, and insertions of genetic material, as well as balanced rearrangements, such as inversions and interchromosomal and intrachromosomal translocations. Additionally, SVs include mobile element insertions, multi-allelic CNVs of highly variable copy number, segmental duplications and complex rearrangements that consist of multiple combinations of these described events. SVs are present in every human genome and affect molecular and cellular processes, regulatory functions, 3D structure and transcriptional machinery. As a result, individual algorithms and single molecular strategies for SV detection may be better suited for some SVs and less for others. The complexity and variety of SVs present numerous challenges for not only accurate detection, but for benchmarking as well, which may have adverse implications to the reliability and reproducibility of downstream secondary and tertiary analyses.

Accordingly, while de novo assembly and assembly-based SV calling is discussed in the context of certain applications, the implementation of systems and methods of the present disclosure in a read-alignment workflow is consistent with the present disclosure. Likewise, while example workflows illustrate the use of particular software or file types, use of alternative or additional software and file types is also consistent with the present disclosure.

### INTRODUCTION

A DNA molecule is composed of two complementary forward (sense) and reverse (antisense) strands of polynucleotides. The forward or sense strand of a DNA molecule presents a string of quaternary code based on four nucleobases: adenine (A), cytosine (C), guanine (G), and thymine (T). This coded string is structured into different segments certain of which, known as genes, encode the structure of proteins and other important functional molecules of all living organisms.

Most organisms possess multiple genomic DNA (gDNA) molecules in each of their cells which are referred to as chromosomes. The complete set of chromosomes representing an organism's entire genetic information is referred to as its genome. The human genome contains approximately 3.1 billion nucleotide pairs (or base pairs) distributed across two (diploid) sets of 23 chromosomes, one set of homologues from each parent. The full diploid human genome comprises 46 chromosomes made up of 44 autosomes (two of each paternal type) and two sex chromosomes (either XX or XY).

Although human genomes are highly similar, there are millions of differences—termed variants—between different individuals. The different alternative forms of a gDNA sequence that originate from germline (heritable) variants are termed alleles. A set of closely linked alleles that are inherited together from one or the other parent is known as a haplotype. Alternative forms of gDNA sequence caused by environmental conditions or somatic cell division during the life of an individual are termed somatic variants. Genome studies generally center on heritable variants of the germline and thus biological sampling for studying the genome are commonly obtained from germ cells of the individual; whereas oncology studies, for example, may sample both somatic and germ cells of the individual to determine the variant origins of cancers.

Variants may be categorized into different classes according to their properties. For example, variants may be categorized according to their size. The smallest (and most frequent) class are single nucleotide variants (SNVs), which result from changes only to single nucleobases. The second smallest class of variants are short insertions and deletions (indels). Insertions refer to extraneous nucleobases present in an individual genome but absent from a consensus genome representing the wider population (a reference genome), whereas deletions refer to the inverse case of nucleobases missing from the individual genome that are present in a reference genome. A third class of variants in the human genome are structural variants (SVs), which are generally much larger in size than SNVs and indels and oftentimes represent major rearrangements in the structure of the genome. SVs are the most diverse class of variants encompassing a wide variety of large-size genomic rearrangements.

SVs may also be categorized according to copy number. For example, SVs may be co-classified as either copy-number variants (CNVs) (unbalanced) or balanced variants. CNVs include insertions, duplications, or deletions that either add or remove genetic material in a way that affects the number of copies (or dosage) of a gene. By contrast, balanced variants, such as inversions and translocations, merely change the orientation, order, or location of genomic segments, and not the content. Although they do not add or remove any genetic material they have the potential to disturb the gene structure and regulatory landscape of the genome causing phenotypic changes.

All variants may also be categorized according to zygosity. Zygosity denotes the presence of a variant in one or both corresponding alleles of respective homologues of a chromosome pair. Variants that are present in only one of the two alleles are called heterozygous, with one mutant-type (variant) and one wild-type (normal) allele. Variants present in both corresponding alleles are called homozygous. The zygosity of a variant determines the genotype of an individual at a given allelic loci.

All variants may also be categorized as either common or rare according to their frequency. The majority of variants in an individual genome are common (e.g., present in more than 1% of the population of individuals) and are therefore referred to as polymorphisms. For example, common SNVs may be referred to as single nucleotide polymorphisms (SNPs).

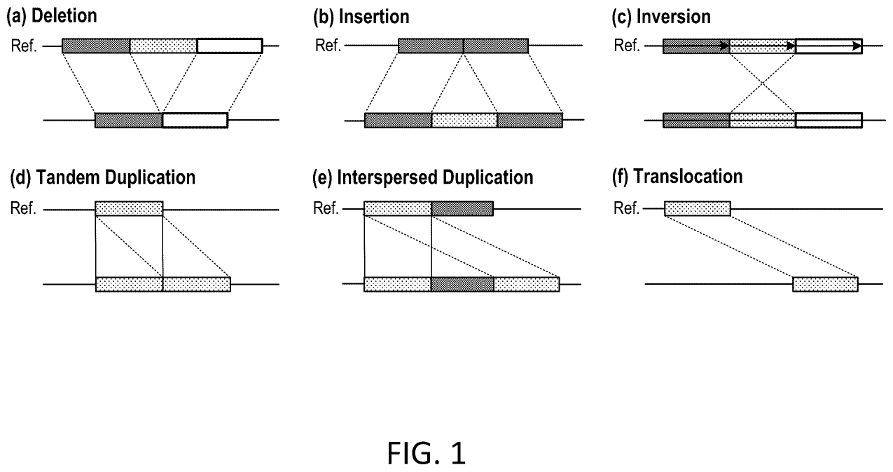

SVs may be classified as either canonical or complex. Referring to FIG. 1, there are generally six canonical classes of SVs: deletions (a), insertions (b), inversions (c), tandem duplications (d), interspersed duplications (e), and translocations (f). A deletion (a) refers to the absence of a genomic segment in an individual genome that is present in the reference genome, which may also be categorized as a CNV. An insertion (b) refers to a genomic segment in an individual genome that is not present in the reference genome at a corresponding locus, which may also be categorized as a CNV. An inversion (c) refers to the reversal of a genomic segment in the individual genome, where the sequence of the segment of the reference genome is present in reverse order at the corresponding locus of the individual genome. A tandem duplication (d) refers to the duplication, in an individual genome, of a genomic segment adjacent the locus of the genomic segment in the reference genome, which may also be categorized as a CNV or as a type of insertion. An interspersed duplication (e) refers to the duplication, in an individual genome, of a genomic segment inserted a distant location from the locus of the genomic segment in the reference genome. The duplication can reside either within the source chromosome (intra-chromosomal) or in a different chromosome (inter-chromosomal). Interspersed duplications are a type of CNV and can also be regarded as a special type of insertion. A translocation (f) refers to a change in position of a genomic segment, either intra-chromosomal or inter-chromosomal. The most common type of translocation is reciprocal, in which genomic segments are exchanges between two chromosomes.

Complex SVs are complex or nested rearrangements that are typically composed of three or more breakpoint junctions and cannot be characterized as a single canonical SV type. Examples include paired-duplication inversion (dupINVdup), paired-deletion inversion (delINVdel), paired-deletion/duplication inversion (delINVdup dupINVdel), deletion-flanked inversion (delINV INVdel), insertion with insersion-site deletion (dDUP-iDEL INS-iDEL), duplication-flanked inversion (dupINV INVdup) and dispersed duplication (dDUP).

The gains, losses, and reorganizations of genes and regulatory elements by SVs impact phenotype and cause diseases. For example, dosage changes to genes by unbalanced SVs can lead to a significant number of autosomal dominant, autosomal recessive, X-linked, and complex trait diseases. Reorganization of DNA content by balanced SVs can result in gene fusions that express chimeric proteins associated with pediatric cancers and liquid tumors. Reorganization also interferes with interactions between genes and regulatory elements, which can lead to complex disorders. For example, SVs in cis-regulatory elements have been associated with autism.

### Terminology and Concepts

As used herein, a Genome is the complete hereditary information of an organism encoded in its DNA. Genomic DNA (gDNA) refers to DNA of a genome.

As used herein, a DNA sequence refers to a sequence or subsequence of nucleotides of a DNA molecule, were each nucleotide in a sequence may be identified by its nucleobase-A, G, C, or T.

As used herein, a base pair (bp) is a unit of measure (length) equal to a single nucleotide position of a DNA sequence. A kilobase (kb) is equal to 1000 bp and a Megabase (Mb) is a 1,000,000 bp measure.

As used herein, a sample nucleotide sequence refers to a sequence of nucleotides isolated or extracted from a sample organism (or a copy of such an isolated or extracted sequence). A nucleotide sequence includes a segment of a nucleic acid polymer that is isolated or extracted from a sample organism and composed of nitrogenous heterocyclic bases. For example, a nucleotide sequence can include a segment of deoxyribonucleic acid (DNA), ribonucleic acid (RNA), or other polymeric forms of nucleic acids or chimeric or hybrid forms of nucleic acids noted below. More specifically, in some cases, the sample nucleotide sequence is found in a sample prepared or isolated by a kit and received by a sequencing device.

As used herein, DNA sequencing is a process of inferring (or base calling) a sample nucleotide sequence of a DNA molecule. A read is an inferred sequence of nucleotides determined in a sequencing process. A read length is the length (bp) of a read used in a particular sequencing process. Reads may generally be categorized as short reads <1 Kb or long reads ≥1 Kb. Short reads include single-end reads (˜150-300 bp) and paired-end reads (˜300-800) obtained using a sequencing-by-synthesis (SBS) process (Illumina, Inc.). Long reads include linked reads (˜2-5 kb) obtained using a mate pair sequencing process (Illumina, Inc.), circular consensus sequencing (CCS) reads (˜13-16 kb) using a HiFi sequencing process (PacBio), or continuous reads (˜30-40 kb) using a nanopore sequencing process (Oxford Nanopore Technologies (ONT)). Given the ˜3.1 billion bp. of the human genome (and read depth requirements discussed herein), along with read depth requirements discussed herein, the sequencing of an individual genome may result in 10 s of millions of reads.

As used herein, a haplotype (H) is one of two DNA molecules (H1 and H2) of a chromosome (or sequence of a region or segment of a chromosome) that includes one of two sets of paired alleles inherited together from one or the other parent. Genotype encompasses both sets of paired alleles. In that regard, genotype reflects the presence of SVs in one (heterozygous) or both (homozygous) alleles.

As used herein, read depth is the number of unique reads that include a nucleotide at a single position in the reconstructed sequence averaged across the sequence. Read depth increases with the number of times a genomic sample is sequenced and thus can be calibrated for objective during sequencing. For example, Read depth may be used to correct random (non-bias) sequencing errors and to distinguish sequencing errors from small variants, e.g., SNPs. In graph-based de novo assembly, increasing read depth may ensure saturation of the graph (e.g., de bruijn or overlay graph) and increase the size of contig assemblies.

As used herein, a k-mer (or k tuple or k-string) is a string of consecutive k nucleotides of a sequence of N length (or I interval), where 1≤k≤N and each k-mer is obtained by sliding a window of k length through the sequence one nucleotide position such that N−k+1 number of overlapping strings is obtained. For example, the sequence AAGTCCGTAGACTA (N=9) at k=3 yields 7 k strings AAG/AGT/GTC/TCC/CCG/CGT/GTA, (k₁₋₇) where each preceding k-string is aligned at two positions with a following k-string. The alignments may be used as nodes (each node a suffix for a preceding k-string and prefix for a following), e.g., in graph-based assembly methods described herein.

As used herein, a trio is a set of reads of respective child (CH) (or proband), maternal (M), and paternal (P) genomes (together, parental or pedigree read data), where trio haplotypes may be expressed as CHH1/CHH2, MH1/MH2, and PH1/PH2 and haplotype heredity of the transmitting parent may be expressed as M-CHH1/P-CHH2.

As used herein, a contig is a contiguous sequence generated from determining the non-redundant path along an order set of component sequences (e.g., reads). A scaffold is an order set of contigs.

As used herein, an assembly is a set of chromosomes, unlocalized and unplaced (random) sequences, and alternate loci used to represent an organism's genome. A chromosome assembly is a relatively complete pseudo-molecule assembled from smaller sequences that represent a biological chromosome. And a diploid assembly is a genome assembly for which a chromosome assembly is available for both sets of an individual's chromosomes.

As used herein, depth of coverage may be measured by the length of sequenced fragments (L) multiplied by the number of assembled fragments (N), divided by the sizes of the genome (G).

A reference genome as referred to herein may comprise sequence data of whole genome reference assemblies, e.g., NCBI36/hg18 or GRCh37/hg19. Alternatively or additionally, a reference genome may include genome sequence data, including sequence annotations and data reports, from public repositories including, e.g., GenBank, NCBI, dbEST, dbSTS, EMBL, and the DDBJ.

As used herein, the term “genomic coordinate” refers to a particular location or position of a nucleotide base within a genome (e.g., an organism's genome or a reference genome). In some cases, a genomic coordinate includes an identifier for a particular chromosome of a genome and an identifier for a position of a nucleotide base within the particular chromosome. For instance, a genomic coordinate or coordinates may include a number, name, or other identifier for a chromosome (e.g., chr1 or chrX) and a particular position or positions, such as numbered positions following the identifier for a chromosome (e.g., chr1:1234570 or chr1:1234570-1234870). Further, in certain implementations, a genomic coordinate refers to a source of a reference genome (e.g., mt for a mitochondrial DNA reference genome or SARS-CoV-2 for a reference genome for the SARS-CoV-2 virus) and a position of a nucleotide base within the source for the reference genome (e.g., mt:16568 or SARS-CoV-2:29001). By contrast, in certain cases, a genomic coordinate refers to a position of a nucleotide base within a reference genome without reference to a chromosome or source (e.g., 29727).

As used herein, the term “sequence file” may include binary base call (BCL), FASTA, and FASTQ files for base call outputs, including binary counterparts to FASTA and FASTQ files, or any another sequencing data format that is capable of being recognized for processing. A FASTQ file may include a text file that contains sequence data from clusters that pass filter on a flow cell. A FASTQ file may include the text file along with corresponding quality scores of the sequence. For simplicity, “FASTA” as used in examples herein may refer interchangeably to FASTA or FASTQ text files, as well as binary counterparts to FASTA and FASTQ files, as appropriate.

As further used herein, the term “alignment score” refers to a numeric score, metric, or other quantitative measurement evaluating an accuracy of an alignment between a nucleotide read or a fragment of a sample nucleotide sequence and another nucleotide sequence from a reference genome. In particular, an alignment score includes a metric indicating a degree to which the nucleobases of a nucleotide read match or are similar to a reference sequence or an alternate contiguous sequence from a reference genome. In certain implementations, an alignment score takes the form of a Smith-Waterman score or a variation or version of a Smith-Waterman score for local alignment, such as various settings or configurations used by DRAGEN by Illumina, Inc. for Smith-Waterman scoring.

An “alignment file” refers to a digital file with an alignment format for storing a representation of read or assembly sequence alignments to a reference sequence. Alignment files may include digital files in Sequence Alignment/Map (SAM), Binary Alignment Map (BAM), or Compressed Reference-oriented Alignment Map (CRAM) file formats. A SAM file is a human-readable text file for storing indexed sequence alignments and a BAM file is its binary equivalent. A CRAM file is a more highly-compressed alternative to BAM file formats that uses a column-oriented binary container format. Alignment files may further include digital files in a pileup format for storing text pileup output of sequence alignments from one or more BAM-files. Alignment files may also include digital files in Browser Extensible Data (BED) file format, including ENCORE BED variant formats. The BED format is a tab-delimited text file format used to store region assemblies as genomic coordinates and associated annotations. Alignment files may also include, e.g., multiple alignment formats (MAF) files, bigMAF format files, Tiling Path Files (TPF), chain format files, General Feature Format (GFF) files, Gene Transfer Format (GTF) files, HDF5 format files, longrange track format files, microarray format files, axt alignment format files, PSL track format files, wiggle track format files, bigBed track format files, bigGenePred track format files, bedGraph format files, and big Lolly format files.

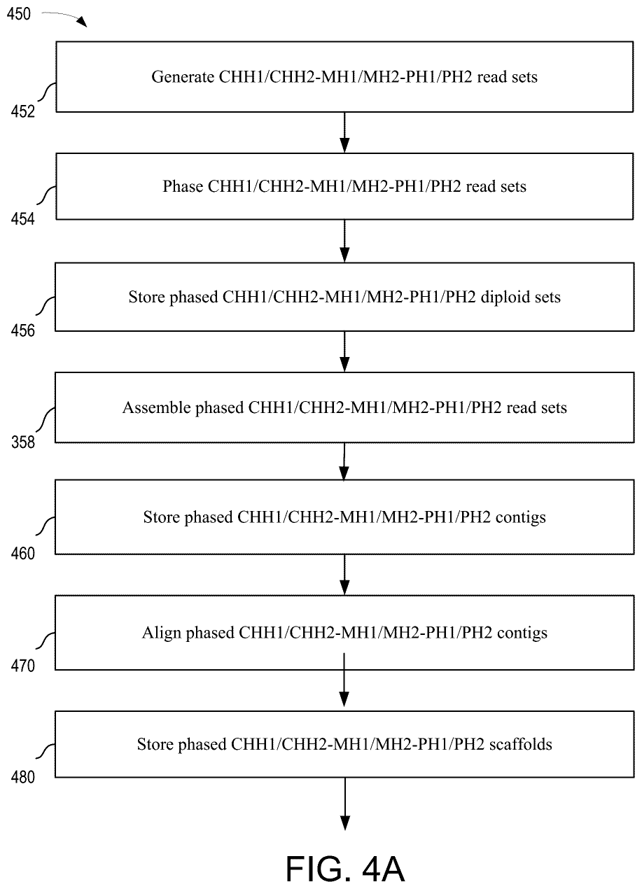

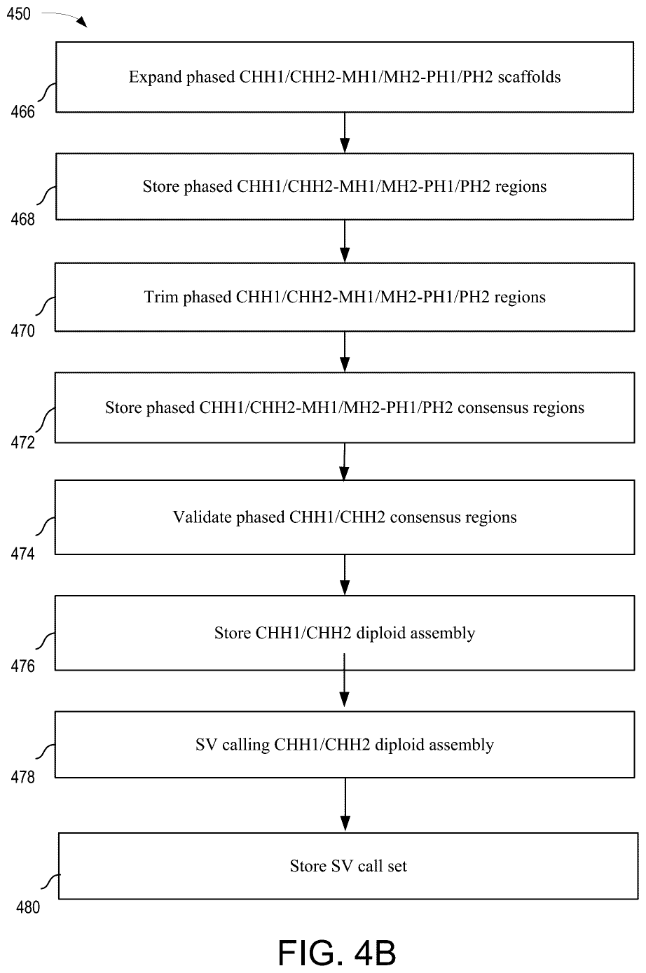

As further used herein, a variant call format (VCF) is a standardized text file format used for representing SNP, indel, and SV calls, and includes its binary counterpart BCF. A VCF file may include two main sections: the header, and site records for each variant call. The header may contain information about the dataset and relevant reference, as well as definitions of all the annotations used to qualify and quantify the properties of the variant calls contained in the VCF file. Generally, the header includes FILTER, FORMAT, and INFO lines. The FILTER lines may identify the filters that have been applied to SV calls emitted to lines of the VCF. For example, the example VCF file of FIG. 4C provides the following filter: ##FILTER=<ID=DUP_AS_INS,Description=“Original INS record that has been converted to DUP”>. Records that fail that filter will contain the ID of the filter, here “Original INS record that has been converted to DUP.”

For each site record, the information on a variant call is organized under columns (or fields), each representing a property observed at the level of the variant site. They most commonly include the fields #CHROM and POS, for the contig and genomic coordinates at which the variant occurs; ID, for identification of the variant, REF and ALT, for reference and alt alleles; QUAL, for Phred-scaled probability that a REF/ALT polymorphism exists at this site given sequencing data, FILTER, for the names of any filters that the variant fails to pass, or a value [PASS] if the variant passed all filters; and INFO, for annotations of context information represented as tag-value pairs or by an ID. The site records may also contain a column for genotype (GT), allele depth (AD), depth of coverage (DP), genotype quality (GQ), and Phred-scaled likelihoods (PL).

There are generally two VCF version in use: versions 4.2 and 4.4. VCF v4.4 may include a StructuralVariantAnnotation package, which includes a unified representation format for harmonizing SV data across different software output formats.

The “mapping quality” or the “mapping quality score (MAPQ)” of a given read alignment quantifies the probability that its position on the genome is correct. The mapping quality is encoded in the PHRED scale where P is the probability that the alignment is not correct. The probability may be calculated as: P=10^{-MAQ/10}, where MAQ is the mapping quality. For example, a mapping quality of 40=10 to the power of −4, meaning that there is a 0.01% chance that the read was aligned incorrectly. The mapping quality is therefore associated with several alignment factors, such as the base quality of the read, the complexity of the reference genome, and the paired-end information. Regarding the first, if the base quality of the read is low, it means that the observed sequence might be wrong and thus its alignment is wrong. Regarding the second, the mappability refers to the complexity of the genome. Repeated regions are more difficult to map and reads falling in these regions usually get low mapping quality. In this context, the MAPQ reflects the fact that the reads are not uniquely aligned and that their real origin is difficult to determine given the limits of information obtainable from sequencing data. Regarding the third, in case of paired-end sequencing data, concordant pairs are more likely to be well aligned. The higher the mapping quality, the better the alignment. A read aligned with a good mapping quality usually means that the read sequence was good and was aligned with few mismatches in a high mappability region. The MAPQ value can be used as a quality control of the alignment results. The proportion of reads aligned with an MAPQ higher than 20 is usually for downstream analysis.

As used herein, a patch is an accessioned scaffold sequence that represents a reference assembly update. Patches add information to the assembly without disrupting the chromosome coordinates and are given chromosome context via alignment to the subject assembly. Together, the scaffold sequence and alignment define the patch. There are two types of patches, novel patches and fix patches. Novel patches represent the addition of new alternate (ALT) loci to the assembly and may be aligned to the existing reference sequence as accessioned scaffold sequences. Fix patches represent changes to existing assembly sequences, either error corrections or improvements such as extensions into gaps.

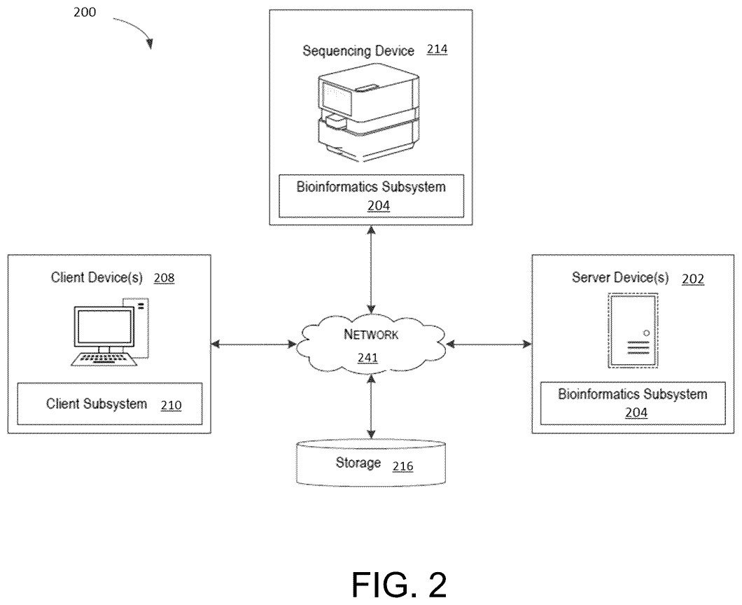

FIG. 2 illustrates a schematic diagram of a system environment (or “environment”) 200 for doing xyz—RA, as described herein. As illustrated, the environment 200 includes one or more server device(s) 202 connected to one or more client device(s) 208 and a sequencing device(s) 214 via a network 212.

As shown in FIG. 2, the server device(s) 202, the client device(s) 208, and the sequencing device(s) 214 may communicate with each other via the network 212 or directly. The network 212 may comprise any suitable network over which computing devices can communicate. The network 212 may include a wired and/or wireless communication network. Example wireless communication networks may be comprised of one or more types of radio frequency (RF) communication signals using one or more wireless communication protocols, such as a cellular communication protocol, a wireless local area network (WLAN) or WIFI communication protocol, and/or another wireless communication protocol. In addition, or in the alternative to communicating across the network 212, the server device(s) 202, the client device(s) 208, and/or the sequencing device(s) 214 may bypass the network 212 and may communicate directly with one another.

As further illustrated in FIG. 2, the environment 200 may include storage 216. The storage 216 can store information for being accessed by the devices in the environment 200. The server device(s) 202, the client device(s) 208, and/or the sequencing device(s) 214 may communicate with the storage 216 (e.g., directly or via the network 212) to store and/or access information.

The sequencing device(s) 214 may comprise one or more devices for sequencing a polynucleotide sample. The polynucleotide sample may include human or non-human deoxyribonucleic acid (DNA) or ribonucleic acid (RNA) analytes, including complimentary nucleotide sequences, coding and non-coding sequences, polynucleotide conjugates and analogues, synthetic and recombinant polynucleotides, and amplicons or clones thereof. DNA samples herein may include sample genomic DNA (gDNA) of various ploidy, including gDNA obtained from the diploid genome of humans. Depending on the desired sequencing data and technique employed to obtain the data, the polynucleotide sample may be obtained from single-cell sampling, clonally expanded cell cultures, bulk sampling of tissues; may be obtained from somatic cells, germ line cells, or combinations of individual somatic and germ line cells, may be obtained from healthy or normal cells, diseased or abnormal cells, or cells having (or lacking) a particular phenotypic trait, genomic marker, or mutagenicity. Sequencing data may include data obtained from gene panels, whole genomic sequencing (WGS), whole exome sequence (WES) data, sequencing a chromosomal region of interest, including coding and non-coding regions, haploid, diploid or polyploid sequencing, single individual (proband) genomic sequencing or proband and corresponding pedigree sequencing, e.g., trios.

The sequencing device(s) 214 may use any number or combination of sequencing techniques consistent with the disclosure. For example, some sequencing techniques consistent with the present disclosure obtain sequence information from a genomic sample through sequencing of small DNA fragments of the sample as short reads, which may be broadly defined to refer to read of less than 1 kb length. For example, an Illumina sequencing platform, e.g., Illumina NovaSeq 6000 system, performs massively parallel, high-throughput WGS using sequence-by-synthesis (SBS) and reverse terminator-based sequencing chemistry to generate reads in the range of ˜150-300 bp length. Sequencing workflow on the NovaSeq platform generally involves loading DNA library onto a flow cell and hybridizing individual fragments to adapter-specific complimentary oligonucleotides (oligos) covalently bound to solid support structures on the flow cell surface; clustering the individual fragments into thousands of identical DNA template strands (amplicons) through bridge or exclusion amplification; and, finally, sequencing, in which copy strands are simultaneously synthesized and sequenced on the DNA templates using reversible terminator-base process wherein within each synthesis cycle fluorophore-labeled single bases are detected through induced fluorescence as they added to the copy strands and then replaced with non-labeled analogues before the start of each subsequent cycle. Because the multiple template strands of each cluster have the same sequence, base pairs incorporated into the corresponding copy strands in each round will be the same, and thus the signal generated from each round will be enhanced proportional to the number of copies of the template strand in the cluster.

Larger reads in the range of ˜300 to 800 bp may be obtained using an SBS-based technique as described above using paired-end sequencing chemistry to generate paired-end reads of each fragment in both forward and reverse directions. Thus, for example, continuous reads may be generated for 300 bp fragments using a 150 bp cycle kit, for 600 bp fragments using a 300 bp cycle kit, and so on. Still larger reads may be generated through computational leveraging. For example, reads may be generated for 800 bp fragments using a 300 bp cycle kit by inserting a known length between the paired ends (for simplicity, a 200 bp insert corresponding to the delta between the 800 bp fragment and the 2×300 bp paired end reads) and inferring the sequence of the insert from the intersection of aligned read data in a pileup format. In one example, long insert paired-end reads is generated in combination with short insert paired reads sequenced at higher depth to infer long insert sequences.

In addition to short-read sequencing, the sequencing device(s) 214 may use any number or combination of long-read techniques to generate read data consistent with the present disclosure. For example, long-read sequencing may be performed using SBS-based sequencing on e.g., Illumina NovaSeq 6000 system, using mate pair sequencing chemistry that enables the generation of reads in the range of several kilobases (Kb), such as Illumina Complete Long Reads (ICLR). Here, the sample gDNA may first be tagmented at desired fragment lengths with a Mate Pair Tagment Enzyme, which attaches a biotinylated junction adapter to each end of the tagmented molecule. The tagmented DNA molecules may then be circularized and the ends of the genomic fragment linked by the respective biotin junction adapters. Circularized molecules may then be re-fragmented yielding smaller fragments suitable for amplification and sequencing. Sub-fragments containing the original junction may then be enriched via the biotin tag in the junction adapter. After End Repair and A-tailing, TruSeq DNA adapters are then added, enabling amplification and sequencing. The short, fragmented reads may then be aligned to yield a long read for the tagmented fragment.

Long-read sequencing may also be performed using circular consensus sequencing (CCS) reads using the single molecule, real-time (SMRT) sequencing technology of Pacific Biosciences. In SMRT sequencing, the continuous incorporation of dye-labeled nucleotides is imaged during DNA synthesis. Single DNA polymerase molecules are attached to the bottom surface of individual zero-mode wavelength detectors (ZMW detectors) that obtain sequence information while phosphor-linked nucleotides are being incorporated into the growing primer strand. The process is repeated to provide a sequence. In one example, CCS sequencing is performed using a HiFi sequencing technique and reagent kit, which provides read depth calibrated to the relatively high random error rates associated with CCS reads.

Long-read sequencing may also be performed using nanopore sequencing. Nanopore sequencing DNA analysis techniques are developed by a number of companies, including, for example, Oxford Nanopore Technologies (Oxford, United Kingdom), Sequenom, NABsys, and the like. Nanopore sequencing is a single-molecule sequencing technology whereby a single molecule of DNA is sequenced directly as it passes through a nanopore. A nanopore is a small hole, typically of the order of 1 nanometer in diameter. Immersion of a nanopore in a conducting fluid and application of a potential (voltage) across it results in a slight electrical current due to conduction of ions through the nanopore. The amount of current that flows is sensitive to the size and shape of the nanopore. As a DNA molecule passes through a nanopore, each nucleotide on the DNA molecule obstructs the nanopore to a different degree, changing the magnitude of the current through the nanopore in different degrees. Thus, this change in the current as the DNA molecule passes through the nanopore provides a read of the DNA sequence.

The sequencing device(s) 214 may also perform orthogonal sequencing techniques, e.g., Chromosome Conformation Capture (Hi-C), Strand-seq. Hi-C involves sequencing crosslinked chromatin to provide information about DNA sequences that may be distant in the linear genome but proximal in 3D space. Hi-C read pairs can span megabases, making the method useful for mapping other read data for detecting large SVs, especially translocations. Strand-seq is a single-cell sequencing process that preserves structural contiguity of individual homologs in single cell samples. Strand-seq technique uses a thymidine analog to selectively label and remove a DNA strand (the nascent strand, synthesized during DNA replication), which generates directional sequencing libraries of DNA template strands only. In particular, Strand-seq may be used in combination with phasing and assembly processes herein, e.g., to i) sort reads or contigs by chromosome; ii) order and orient contigs, and iii) obtain chromosome-wide phase signals irrespective of physical distance.

Other example sequencing techniques include, e.g., pyrophosphate sequencing (e.g., Genome Sequencer FLX from 454 Life Sciences), ion-sensitive sequencing (e.g., Personal Genome Machine and Proton from Ion Torrent Systems, Inc.), Helicos True Single Molecule Sequencing (tSMS) technology, chemical-sensitive field effect transistor (chemFET) array, probe-anchor ligation sequencing (e.g., Complete GeGenomics™ or Polonator™), to name a few.

In certain embodiments, sequencing data may be generated from a sample nucleotide sequence using only one sequencing methodology, in which case the environment 200 may include a single sequencing device 214 adapted to perform the particular sequencing methodology, e.g., implementing either a particular short-read or long-read sequencing technique. However, in certain other embodiments, it may be desirable to generate sets of sequencing data from a sample using more than one sequencing technique, e.g., particular short-read and long-read sequencing techniques, in which case the environment 200 may include either multiple sequencing devices 214 for separately generating short-read and long-read sequencing data of a given sample or a single sequencing device 214 adapted with separate chemistries to generate both short-read and long-read sequencing data. For example, Illumina NovaSeq sequencers may generate both short-paired end reads and long linked-end reads, as discussed.

Short reads consistent with the present disclosure may be over 30 bp, over 40 bp, 50-100 bp, 100-200 bp, 25-300 bp. 300-350 bp, 350-400 bp, 400-450 bp, 450-500 bp, 500-550 bp, 550-600 bp, 600-700 bp, 700-800 bp, up to 900 bp, up to 800 bp, up to 700 bp. Long reads for use herein may be over 900 bp, over 1000 kb, over 1500 kb; over 2000 kb; 2000-5000 kb; 5000-8000 kb, 8000-12000 kb, 12000-15000 kb, up to 20000 kb; up to 25000 kb up to 30,000 kb, up to 60,0000 kb, up to 1000000 kb. Read depth for use herein may be 20× to 30×, 50× to 70×, 70× to 100×, 100× to 150× or up to 200×.

As further indicated by FIG. 2, the server device(s) 202 may generate, receive, analyze, store, and/or transmit digital data, such as raw sequencing data for determining nucleotide-base calls or sequencing nucleic-acid polymers. As shown in FIG. 2, the sequencing device(s) 214 may generate and send (and the server device(s) 202 may receive) nucleotide reads and/or other sequencing data for being analyzed by the server device(s) 202 for base calling and/or variant calling. The server device(s) 202 may also communicate with the client device(s) 208. In particular, the server device(s) 202 may send data to the client device(s) 208, including sequencing data or other information and the server device(s) 202 may receive input from users via client device(s) 208.

The server device(s) 202 may comprise a distributed collection of servers where the server device(s) 202 include a number of server devices distributed across the network 212 and located in the same or different physical locations. Further, the server device(s) 202 may comprise a content server, an application server, a communication server, a web-hosting server, or another type of server.

As further shown in FIG. 2, the server device(s) 202 and/or the sequencing device(s) 214 may include a bioinformatics subsystem 204. The bioinformatics subsystem 204 may include software and/or hardware utilized by the server device(s) 202 for processing sequencing requests and/or data, as described herein. The bioinformatics subsystem 204 may be included in a single server device 202 or sequencing device(s) 214, or may be distributed across multiple devices. The bioinformatics subsystem 204 may include a sequencing system that spans multiple layers of software and/or hardware for servicing requests for sequencing services at the bioinformatics subsystem 204. The sequencing services may include primary analysis for generating sequencing data from images, secondary analysis for determining variant calls or other results from the sequencing data, and/or tertiary analysis for further analyzing the results of the secondary analysis to better understand the results, for example.

The bioinformatics subsystem 204 may perform all or a portion of primary data analysis, including, for example, analysis of raw read data (e.g., signal analysis), targeted generation of legible sequencing reads (base calling) and scoring base quality. In addition to performing primary analysis functions, the bioinformatics subsystem 204 may generate files for processing and/or transmitting to other devices for performing secondary and/or tertiary analysis functions.

The bioinformatics subsystem 204 may support any file format supported by the Sequence Read Archives (SRA) at National Center for Biotechnology Information (NCBI) or the human genome browser at UCSC including, e.g., binary base call (BCL), FASTA, and FASTQ files for base call outputs, including binary counterparts to FASTA and FASTQ files, BAM, SAM, CRAM, BED files for aligner and assembler outputs, and VCF, BCF, and gVCF for SV caller outputs. Other supported formats may include, e.g., MAF, TPF, Axt, PSL BED detail, big lolly, bedGraph, bigBed, barChart, bigBarChart, GenePred table, bigGenePred table, bigPsl table, bigMaf table, bigNarrowPeak table, bigLolly table, wiggle track, bigWig, Chain, ENCODE-specific formats, GFF, GTF, HAL (HDF5), Hic, Interact, bigInteract, Longrange longTabix, Microarray, Net, Personal Genome SNP, 2 bit, and nib.

Each client device 208 may generate, store, receive, and/or send digital data. In particular, the client device 208 may receive sequencing metrics from the sequencing device(s) 214. Furthermore, the client device 208 may communicate with the server device(s) 202 to receive one or more files comprising nucleotide base calls and/or other metrics. The client device 208 may present or display information pertaining to the nucleotide-base call within a graphical user interface to a user associated with the client device 208.

The client device(s) 208 illustrated in FIG. 2 may comprise various types of client devices. In examples, the client device 208 may include non-mobile devices, such as desktop computers or servers, or other types of client devices. In other examples, the client device 208 may include mobile devices, such as laptops, tablets, mobile telephones, or smartphones.

As further illustrated in FIG. 2, each client device 208 may include a client subsystem 210. The client subsystem 210 may include software and/or hardware utilized by the client device 208 for processing sequencing requests and/or data, as described herein. The client subsystem 210 may span multiple layers of software and/or hardware. The client subsystem 210 may be included in a single client device 208 or may be distributed across multiple client devices 208.

The client subsystem 210 may comprise a sequencing application. The sequencing application may be a web application or a native application stored and executed on the client device 208 (e.g., a mobile application, desktop application). The sequencing application may include instructions that (when executed) cause the client device 208 to receive data from the sequencing device(s) 214 and/or the server device(s) 202 and present, for display at the client device 208, data to the user of the client device 208.

Client processes may be operated on one or more of the client device 208, the server device 202, and/or the sequencing device(s) 214 for requesting sequencing services from the bioinformatics subsystem 204. For example, client processes executing on any of the client device(s) 208, server device(s) 202 and/or sequencing device(s) 214 may transmit requests for performing secondary analysis and/or tertiary analysis at the bioinformatics subsystem 204. The bioinformatics subsystem 204 may load and/or execute different bitstreams to perform different types of secondary analysis and/or tertiary analysis to support requests from the client processes.

The secondary analysis described herein may result in variant calls and/or variant call files generated from the sequencing data. Variant calling pipelines herein may include small variant calling different variant classes, including small variant calling for identification of single nucleotide polymorphisms (SNPs) or insertions or deletions (indels) of generally 50 bp or fewer; copy number variant (CNV) calling for detection of large insertions and deletions associated with genomic copy number variation of generally from 50 bp to several mb; short tandem repeat (TR or STR) calling for detection of highly polymorphic microsatellites of recurring DNA motifs of ˜2-6 bp, and structural variant (SV) for detection of large complex variant structures generally above 1000 kb, and may include a variety of variant classes, including large insertions and deletions, including CNVs, multi-allelic CNVs, mobile element insertions (MEI), translocations, inversions, duplications. STR and SV variant classes are believed to have a disproportionate effect on gene expression compared to SNVs and indels. SV and STR variant classes have a disproportionate effect on gene expression compared to SNPs and indels. Expansion or contraction within TR segments, in which individual variants is coterminous with the length of the TRs, e.g., ≥˜5 bp, may also be considered CNVs and thus may also be reported (e.g., in VCF 4.4 as <CNV:TR>). However, given complexity and variety these variant classes, STR and SV calling generally implement multiple computational approaches and deep whole genome sequencing to accurately identify and genotype variants in these different classes.

A number of tasks of systems described herein may be offloaded from software to configurable hardware, e.g., FGPAs, for acceleration of computational processing. For example, one or more FPGAs may be configured to perform tasks at various stages of secondary analyses performed herein. For instance FPGA may be parallelized for multithreading, including, e.g., multithreading variant calling by chromosome. FPGAs may be instantiated with graphs or patterns for do novo assembly. And they may be configured for highly repeated processes, such as hidden Markov Models or Smith-Waterman algorithm.

The bioinformatics subsystem 204 may prepare a datafile mapping colinear and orthogonal alignments of phased child (CHH1/CHH2), maternal (MH1/MH2), and paternal (PH1/PH2) contigs (or scaffolds) from a trio dataset. The bioinformatics subsystem 204 may generate corresponding consensus regions from the colinear and orthogonal alignments mapped in the datafile. The bioinformatics subsystem 204 may validate the CHH1/CHH2 consensus regions to based, in part, on composition vector analysis of the parental consensus. The validated CHH1/CHH2 consensus regions may be stored to a BED file as a diploid assembly. And an SV caller subsystem may operate an SV caller engine to perform haplotype-aware variant calling in the diploid assembly.

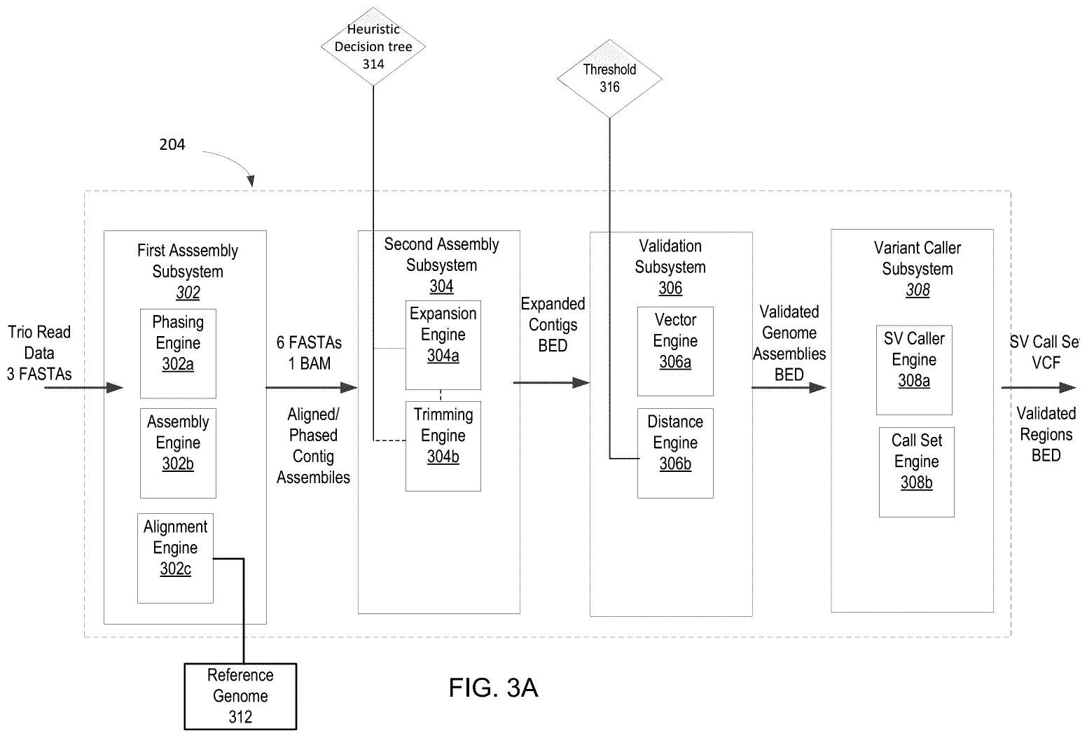

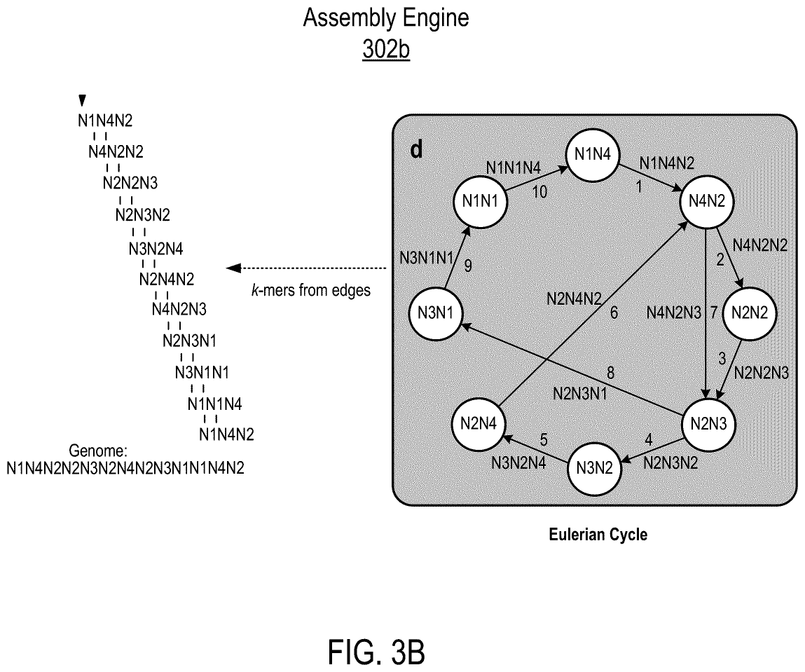

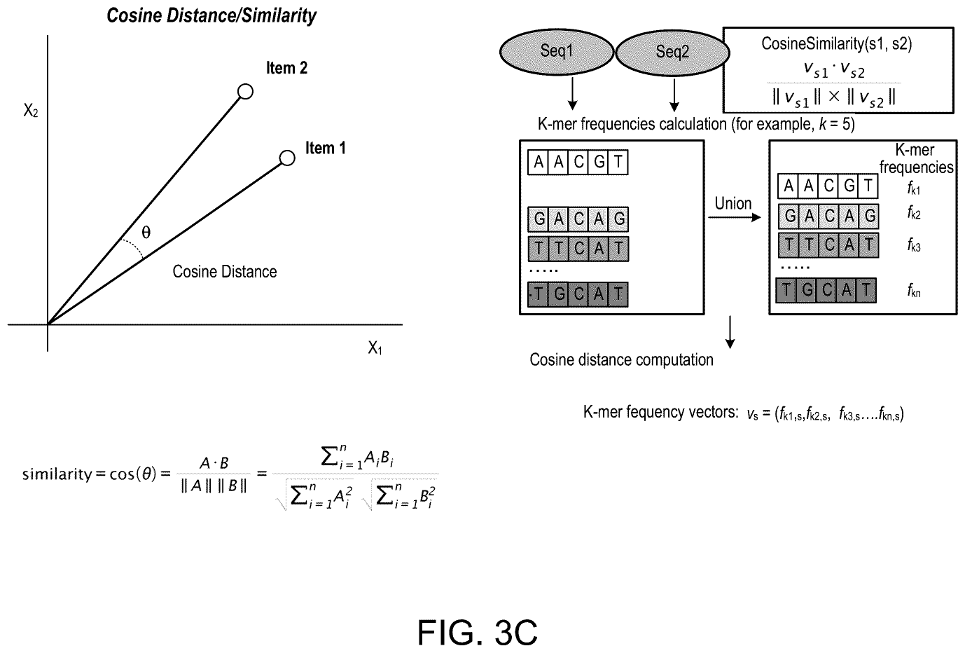

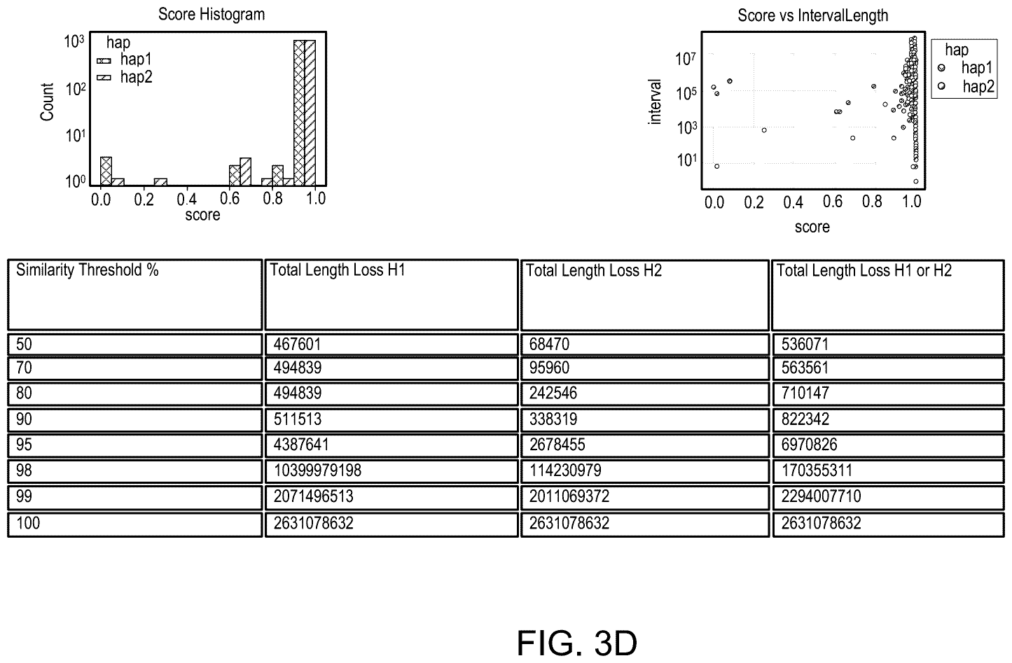

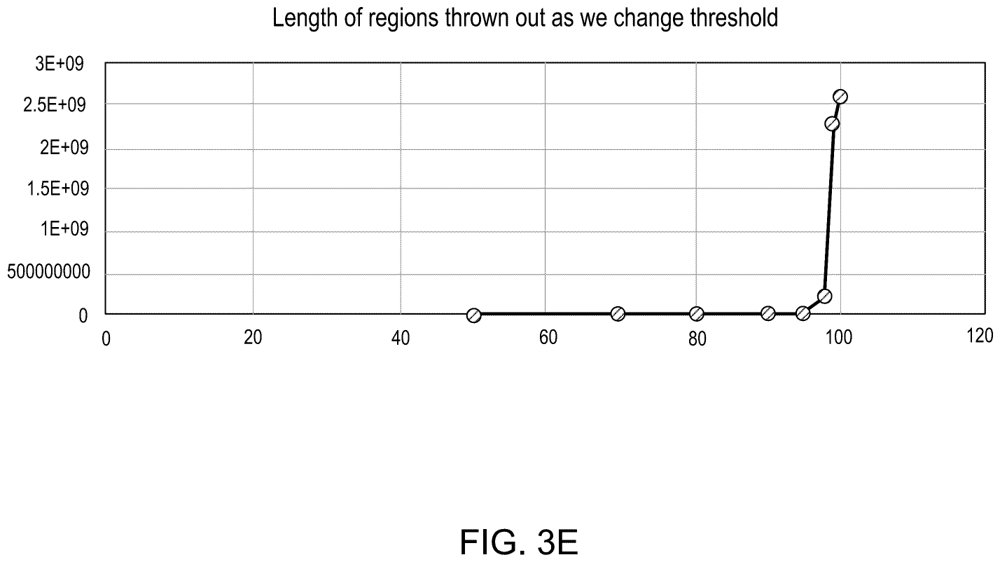

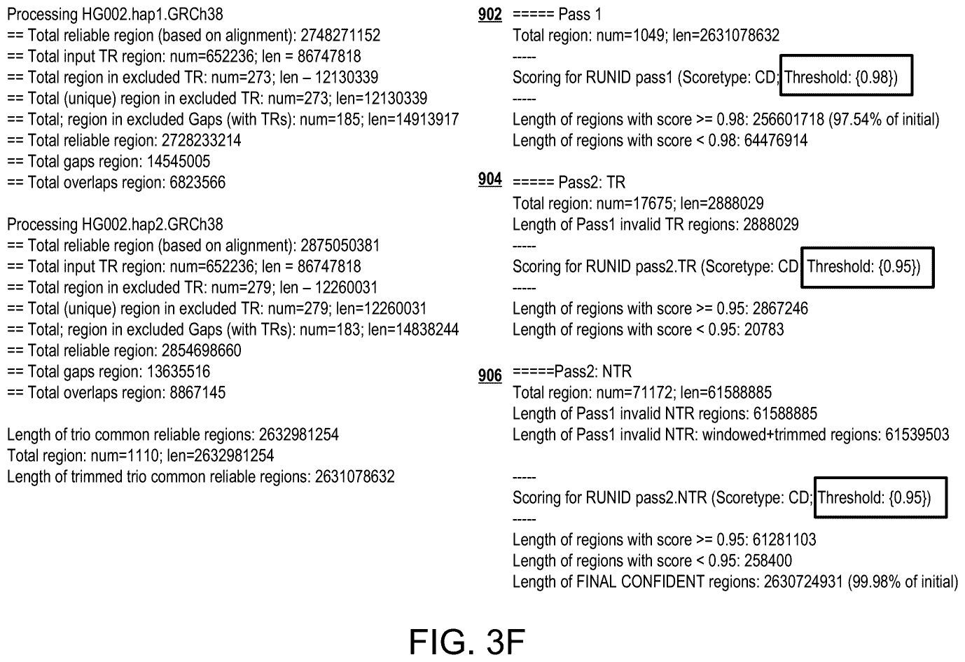

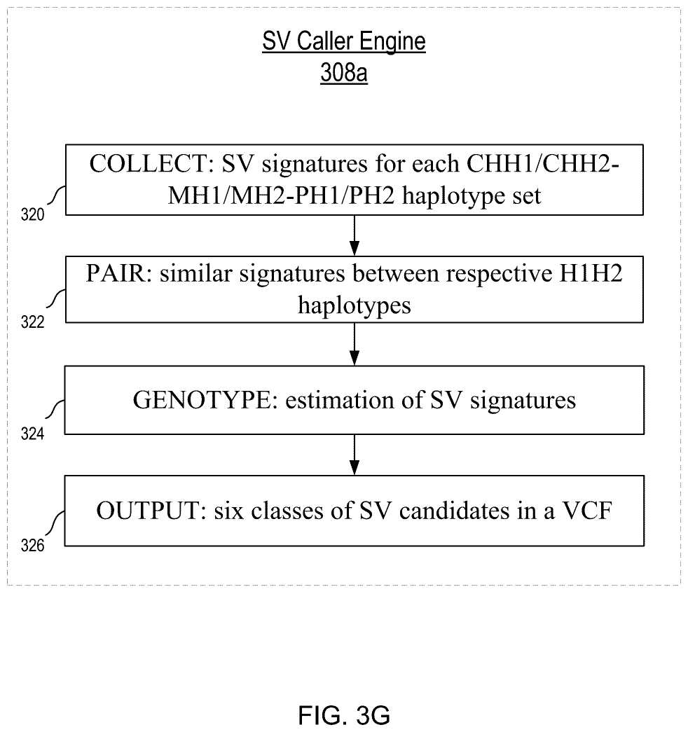

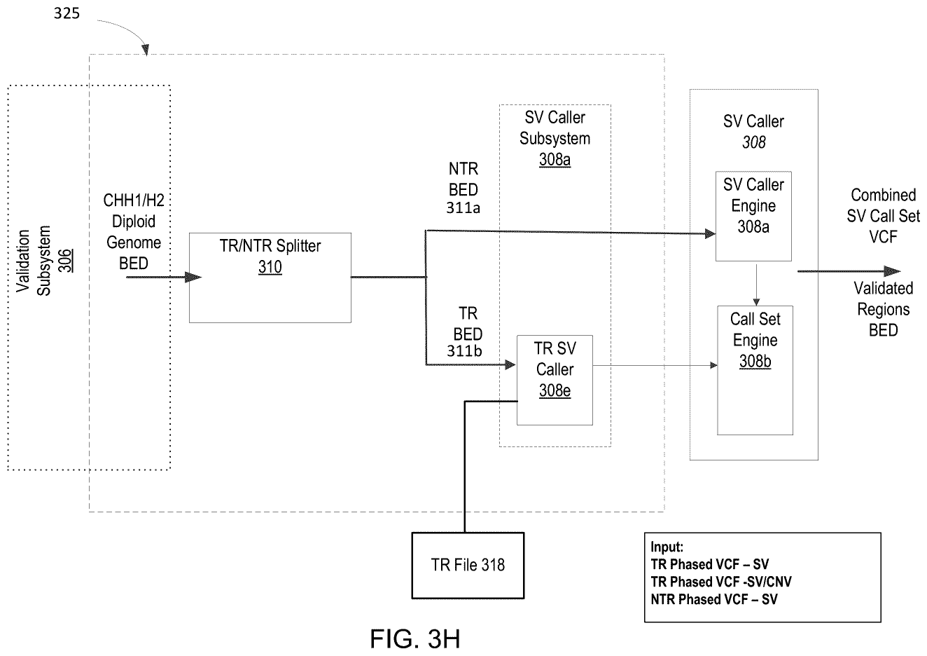

In one aspect, a bioinformatics subsystem 204 may include one or more additional secondary analysis subsystems for implementing one or more methods or procedures described herein. For example, as shown in FIG. 3A, the bioinformatics subsystem 204 may include a first assembly subsystem 302, second assembly subsystem 304, validation subsystem 306, and/or variant caller subsystem 308 for implementing one or more methods or procedures described herein. Each such secondary analysis subsystem may perform a different task or set of tasks. Certain tasks of the secondary analysis subsystem may be agnostic to sequencing methodology and may be performed across sequencing data forms (e.g., short- and/or long-read data forms). Conversely, certain other tasks of the secondary analysis subsystem may be unique to the sequencing data forms. Tasks of the secondary analysis subsystem may be unique or agnostic to particular techniques implementing a variant calling pipeline (e.g., de novo assembly-based or read-based variant calling approaches). Tasks of the secondary analysis subsystem may be unique or agnostic to the variant class being called, with a number of tasks being developed to specifically support haplotype-aware, assembly-based variant calling for SVs, including tasks associated with phasing, assembling, variant calling, genotyping/validation, scoring, and reporting, and other task that may be common to SV calling best practices. Moreover, although the secondary analysis subsystems in the example bioinformatics subsystem 204 of FIG. 3 are interoperably arranged in connection with the implementation of the illustrated methods and/or procedures, any combination of one or more of these subsystems may be implemented in manner consistent with the present disclosure.

In examples, the first assembly subsystem 302 may receive trio read data in a sequence file. In one example embodiment, bioinformatics subsystem 204 may implement an SV calling pipeline through operation of one or more secondary analysis subsystems. In examples, the one or more secondary analysis subsystems can receive a trio dataset of diploid read data of sequenced child and parental genomes, and generate, based on the read data, aligned consensus regions in haplotype-specific (phased) child and parental sets, i.e., sets of CHH1/CHH2-MH1/MH2-PH1/PH2 consensus regions (three diploid sets/six haplotype sequence sets). The one or more subsystems can validate the consensus regions of the child diploid set and output, in an alignment format, a datafile (e.g., a BED file) of validated child consensus regions as a diploid assembly. In one example, validation for each consensus region is determined based on composition vectors of corresponding child and transmitting parent sequences at a given interval (I), e.g., interval sequences of M-CHH1 and either of MH1/MH12 and P-CHH2 and either of PH1/PH2. The one or more subsystems can then analyze the datafile and detect structural variants within the stored diploid assembly and output, in a variant call format, a datafile (e.g., a VCF file) of detected SVs as an SV call set.

FIG. 3A shows an example bioinformatics subsystem 204 including secondary analysis subsystems 302, 304, 306, and 308. As shown, the bioinformatics subsystem 204 may operate via one or more secondary analysis subsystems, such as a first assembly subsystem 302, second assembly subsystem 304, validation subsystem 306, and/or SV caller subsystem 308, to perform secondary analysis on trio read data. Though these subsystems are provided as examples, it should be understood that each subsystem may perform tasks that may be combined in a single subsystem or provided in additional subsystems on the bioinformatics subsystem 204.

One or more of the subsystems may be operated on instrument, e.g., on sequencing device(s) 214, on premises, e.g., server device(s) 202, or over network 212. Each of the one or more subsystems 302, 304, 306, 306, and 310 may include engines for performing operations thereon. Subsystems 302, 304, 306, 306, and 310, and their respective engines, may be operated on a single processor, or across two or more processors. Likewise, one or more of subsystems 302, 304, 306, 306, and 310, and their respective engines, may be implemented in a single executable program. As one example, a number of available software tools may perform both phasing (phasing engine 302a) and assembly functions (assembly engine 302b), e.g., HifiAsm, DipAsm, FALCON-unzip, and Supernova. While respective engines of subsystems 302, 304, 306, and 308 are numbered serially, the engines need not necessarily operate sequentially.

In the illustrated example in FIG. 3A, the first assembly subsystem 302 may prepare one or more datafiles through operation of a phasing engine 302a, assembly engine 302b, and alignment engine 302c. For instance, the phasing engine 302a may partition the trio read sets to phased (haplotype-specific) CHH1/CHH2-MH1/MH2-PH1/PH2 read sets. Using a graph building model, for example, the assembly engine 302b may assemble phased reads of each set into a respective set of contigs. The alignment engine 302c may align the CHH1/CHH2-MH1/MH2-PH1/PH2 contig sets using a reference genome 312 to colinearly align contigs within each set to scaffolds mapped to a reference genome 312 and compress the alignment data to of each set into respective alignment files (e.g., BAM files).

Each engine may implement hardware and/or software for use as described herein. The first assembly subsystem 302 may utilize a phasing engine 302a to partition trio read data into phased CHH1/CHH2-MH1/MH2-PH1/PH2 read sets. For example, phasing engine 302a may partition CHH1/CHH2 reads using a trio binning technique, which establishes phase empirically based on inheritance patterns among trio read data.

Example software tools for performing trio binning include HifiAsm, Trio-sga and TrioCano, each of which include modes adapted to analyzes MH1/MH2 and PH1/PH2 short read sets to bin CHH1/CHH2 long reads according to haplotype. Importantly, by phasing the child genome, the read data can be partitioned into two parental genome datasets that can be independently assembled. Such a procedure is particularly valuable for resolving SVs and its haplotype architecture because structural differences between haplotypes have often led to hybrid representations or collapses in the assembly that do not reflect the true sequence and, therefore, may be biologically meaningless.

Phasing engine 302a may also partition MH1/MH2 and PH1/PH2 read sets. For example the parental read sets may be partitioned using physical phasing method or, alternatively, an orthogonal method, e.g., Hi-C reads in a contact map or Strand-seq data. The phasing engine 302a may leverage example software tools for physical phasing parental read sets, such as Beagle (5.2), and/or ShapeIT, which rely on maximum-likelihood models for phasing; DeepVarient, which uses a classifier model implemented in a convolutional neural network (CNN); or, in example implementations utilizing parental long read data, WhatsHap, using a weighted Minimum Error Correction Problem (wMECP) model. Example phasing software tools using orthogonal methods, include HifiAsm (Hi-C mode), DipAsm, and Falcon Unzip.

The output from the phasing engine 304a may be sent directly to the assembly engine 304b, or the output may be stored to a disk and retrieved by assembly engine 304b. For example, the phasing engine 304a may individually compress each of the phased CHH1/CHH2-MH1/MH2-PH1/PH2 read sets into individual sequence files for storage and/or downstream processing.

The assembly engine 302b may implement hardware and/or software for processing as described herein. For example, the assembly engine 302b may receive the sequence files storing copies of phased CHH1/CHH2-MH1/MH2-PH1/PH2 read sets and assemble the phased reads into respective contig sets. In one example, the assembly engine 302b may implement a de Bruijn graph (dBg), overlay string graph, or other graph-building model to assemble reads into contigs. As illustrated in FIG. 3C, the dBg-based model 340, when executed, first decomposes the reads into k-mers (k=3 in the illustration) and builds a dBg representing all overlapping two bp k-string prefixes and suffixes as nodes, and each k-string giving the prefix and suffix as an edge between the nodes. Once the dBg is saturated with reads, the k-string-length sequences are sequentially assembled (or walked) from suffix to prefix out of the graph based on assumptions that the sequences may be assembled by visiting each edge only once, i.e., a Eulerian cycle, or at least once, a Hamiltonian cycle.

In examples, a dBg-based model may be implemented by assembler software. Software adapted for short read assembly may be selected as appropriate, e.g., Velvet, ABySS, SOAPdenova, SPAdes, and RAMPART. In the same or other examples, a dBg-based model may be implemented in assembler software adapted for long reads, e.g., HiFiAsm, Canu, Flye, wtdbg2, and Shasta. Other graph-based models utilizing an overlay string graph (e.g., overlap/layout/consensus (OLC) graph) may be used consistent with the present disclosure. OLC-based assemblers include SSAKE, SHARCGS, VCAKE, Celera Assembler, Arachne, and PCAP.

In some embodiments, a reference- or read-based or approach or hybrid alignment-assembly strategy may be implemented as a complimentary process. For instance, some or all aspects of a read-based SV calling process may be performed to gain local information relevant to a one or more stages of de novo assembly. In examples, alignment engine 302e initiates a read-based SV calling process. According to an example, alignment engine 302c receives sequence files storing copies of phased CHH1/CHH2 read sets generated by phasing engine 302a, as above. Each read set may be aligned to a reference genome 312 to generate pile-ups of overlapping reads. The reads may then be merged and compressed to a BAM file. The BAM file may be accessed, e.g., by assembly engine 302b to obtain local information relevant to one or more assembly tasks, including, e.g., read graphing, error correction, and gap-filing.

Once assembly is completed, an output of phased CHH1/CHH2-MH11/MH2-PH1/PH2 contig sets from the assembly engine 302b may be sent directly to the alignment engine 302c, or the output may be stored to a disk and retrieved from disk by assembly engine 302b. The assembly engine 302b may compress the contig sets into separate sequence files for storage and/or downstream processing.

The alignment engine 302c may implement hardware and/or software for processing as described herein. The alignment engine 302c may include one or more algorithms configured to align a location of each contig to a location on the reference genome 312. For example, the alignment engine 302c may perform local alignment using, e.g., a Smith-Waterman (SW) algorithm for local alignment, global alignment using, e.g., a Needleman-Wunsch (NW) algorithm, or both. The alignment engine 302c may also perform alignment through dynamic programming methods.

The alignment engine 302c may access a reference genome 312 to colinearly align each contig set into scaffolds and map each scaffold to a copy of the reference genome 312 to one or more alignment files, e.g., BAM file. Consistent with the present disclosure, the reference genome 312 may be implemented in a linear format of haploid primary reference contigs. Alternatively, the reference genome 312 may include a graph of alternative contigs stored in one or more lift-over files reflecting wider allelic variation beyond the primary contigs of the reference.

In one implementation, the alignment engine 302c may perform alignment of the contig sets by generating k-mer seeds from the contig sequence data and looking for matches to the reference genome 312. In one example, reference genome 312 may be indexed to a hash table in support of a seed-chain-alignment methodology. In one such implementation, k-mer subsequences of a reference genome or genomic region of genomic reference genome 312 may be indexed to a hash table having the hash of a subsequence as its key and the locations of indexed subsequences as its value. The contig sequence data may then be queried at coincident k-mer frequency (i.e., seeds) to locate exact matches (or anchors) to the reference. Alignment may then be determined through identification of sets of colinear anchors and scoring the sets based on one or more heuristics.

The alignment engine 302c may perform alignment on the locations of each read with a highest density of seed matches or a density above a threshold when compared to other locations of the contig. The alignment engine 302c may compare each position of a contig against each candidate position of the reference genome. These comparisons may correspond to a matrix of potential alignments between the contig and the reference genome. For each of these candidate alignment positions, the alignment engine 302c may generate scores that may be used to evaluate whether the best alignment passing through that matrix cell reaches it by a nucleotide match or mismatch (e.g., diagonal movement), a deletion (e.g., horizontal movement), or an insertion (e.g., vertical movement). An insertion or deletion may be referenced as an indel. A match between the read and the reference genome may provide a bonus on the score. A mismatch or indel may impose a penalty. The overall highest scoring path through the matrix may be the alignment that is chosen. The values chosen for scores by the read alignment engine 302c may indicate how to balance, for an alignment with multiple possible interpretations, the possibility of an indel as opposed to one or more SNPs, or the preference for an alignment without clipping. It will be understood that the tasks performed by a given engine, such as the alignment engine 302c, may be combined with the tasks performed by another engine in a single engine (e.g., alignment/assembly, alignment/phasing, alignment/SV caller engine, etc.)

In examples, alignment engine 302c operates on an aligner software designed for aligning contigs to reference genome 312, e.g., Minimap2 (mm2), BLAST6, MUMmer7, or SSAHA8. According to the method example of FIGS. 4A-C, alignment engine 302b may operate on an mm2 aligner, which is particularly suited for mapping long read contigs, including mapping noisy DNA loci, locating overlaps between contigs, and aligning larger genome assemblies.

The scaffold sets generated from the alignment engine 302c may be sent directly to the second assembly subsystem 304, or the output may be stored to disk and retrieved from disk by the second assembly subsystem 304. The alignment engine 302c may compress the phased CHH1/CHH2-MH1/MH2-PH1/PH2 scaffold sets into one or more alignment files (e.g., BAM file), or other binary format that is machine-readable.

In the same or another embodiment, the second assembly subsystem 304 may analyze one or more alignment files storing a phased dataset of aligned trio contigs and prepare, from corresponding assemblies of the trio contigs, an alignment file mapping colinear and orthogonal alignments of phased CHH1/CHH2-MH1/MH2-PH1/PH2 consensus regions to a stored reference. In one example, the second assembly subsystem 304 may prepare an alignment file of stored consensus regions through operation of an expansion engine 304a and a trimming engine 304b. For instance, the expansion engine 304a may join adjacent contigs of each colinearly aligned haplotype sequence set into region assemblies according to one or more heuristics, in which, e.g., each of the one or more heuristics is a predicate expression of alignment between two adjacent contigs. The trimming engine 304b may generate a consensus among orthogonally aligned region assemblies (or allelic grouping) by trimming portions of any region assembly that do not align with every other assembly of a respective allelic grouping. The second assembly subsystem 304 may then output the consensus in an alignment file as phased sets of CHH1/H2-MH1/H2-PH1/H2 consensus regions.

The expansion engine 304a may implement hardware and/or software for processing as described herein. In one implementation, expansion engine 304a may join colinearly adjacent contigs based one or more rules in accordance with the NCBI Genome Assembly Model, including rules for full dovetail, half dovetail, contained, and short/blunt (<50 bp) adjacencies. In the same or another implementation, the expansion engine 304a may join adjacent contigs of each of the six scaffolds into regions according to one or more heuristics. For example, the expansion engine 304a may implement a set of rules in a heuristic decision tree, in which, e.g., each heuristic is a predicate expression of alignment between two adjacent contigs.

Expansion engine 304a may compress the alignment data of the phased sets of CHH1/CHH2-MH1/MH2-PH1/PH2 consensus regions into a single BED format file. The BED format may include multiple alignment format (maf) for storing the series of multiple alignments of the sets line-oriented to the reference genome 312. In that regard, alignment data as stored in the BED format may include coaxial alignment data for assembled regions of each haplotype set and orthogonal alignment data of allelic groupings of assembled regions across the haplotype sets. In addition or in the alternative, expansion engine 304a may compress the alignment data of the phased CHH1/CHH2-MH1/MH2-PH1/PH2 regions into individual BAM files, or other binary format that is machine-readable.

The output from the expansion engine 304a may be sent directly to the trimming engine 304b or the output may be stored to disk and retrieved from disk by the trimming engine 304b. The trimming engine 304b may implement hardware and/or software for processing as described herein. In one implementation, the trimming engine 304b may receive the phased sets of trio regions in a single BED file and generate, based on line-ordered alignments of the sets, a consensus among each allelic grouping of haplotype sets of CHH1/CHH2-MH1/MH2-PH1/PH2 regions. For instance, the trimming engine 304b may generate a consensus by trimming portions of any region that do not align with every other region of a respective allelic grouping. The second assembly subsystem 304 may then output the consensus in an alignment file as a phased set of CHH1/H2-MH1/H2-PH1/H2 consensus regions.

The set of trio consensus regions output from the trimming engine 304b may be sent directly to the validation subsystem 306 or the output may be stored to disk and retrieved from disk by the validation subsystem 306. The trimming engine 304b may compress the phased dataset of aligned trio consensus regions in a BED file for storage and/or downstream processing. The validation subsystem 306 may receive the BED file and prepare, based on a composition vector analysis, an alignment file storing a phased set of validated consensus regions of the child genome.

In one example, the validation subsystem 306 may operate through a vector engine 304a and/or a distance engine 304b. Vector engine 304a and distance engine 304b may implement hardware and/or software for processing as described herein. For instance, vector engine 304a may calculate composition vectors of each child consensus region at intervals (I) based on a summing of k-mer frequency vectors, where the value k can be 1≤k≤(I) and a frequency vector (v) is determined for each (I)−k+1 number (n) of k-strings in a given interval (I) to yield v₁₋ₙ k-mer frequency vectors per interval (I).

In one implementation, a child consensus region at a given interval (I) may be determined valid if the composition vectors of each child sequence of respective haplotypes is within a threshold distance measure of the composition vector of one of the two sequences of respective haplotypes of a transmitting parent (i.e., between M-CHH1-MH1/MH2, P-CHH2-PH1/PH2). For instance, in one example, the distance measure may be an angle-based measure expressed as a similarity value between 0 and 1, given by Equation 1 of FIG. 3C:

$$similarity = cos ( θ ) = \frac{A \cdot B}{‖ A ‖ ‖ B ‖} = \frac{{\sum }_{i = 1}^{n} {A}_{i} {B}_{i}}{\sqrt{{\sum }_{i = 1}^{n} {A}_{i}^{2}} \sqrt{{\sum }_{i = 1}^{n} {B}_{i}^{2}}} \qquad Equation ( 1 )$$

where:
A is the composition vector at a sequence one,
B is the composition vector at a sequence two,
A·B is a dot product of k-mer frequency vectors Av₁₋ₙ at sequence one and corresponding k-mer frequency vectors Bv₁₋ₙ at sequence two,
∥A∥ ∥B∥ is the Euclidean norm, sequence one is a haplotype-specific sequence of the child consensus region, and sequence two is one of the two haplotype-specific sequences of the transmitting parent.

In certain examples, a child consensus region at a given interval can be determined valid if the composition vector of each child sequence of respective haplotypes is within a threshold distance measure of the composition vector of one of the two sequences of respective haplotypes of a transmitting parent. For instance, a child consensus region at a given interval (I) can be determined valid if the similarity value for each sequence of respective haplotypes of the one of the two sequences of respective haplotypes of a transmitting parent meets a threshold 316. The threshold 316 may be a cosine similarity value between 0 (at which vectors are orthogonal) and 1 (at which vectors are identical). In examples, threshold 316 may be selected based on balancing considerations of sequence loss. For instance, FIGS. 3D-E presents results of testing sequence loss at different thresholds between 50% and 90% at a fixed N and k values testing for loss of total sequence length as a variable. As shown, no appreciable difference in loss was found between thresholds selected at ˜50% and at ˜90%, suggesting that thresholds at ≤90% would generate a high percentage of false positives, whereas the loss between thresholds selected at ˜98% and ˜99% increased by factor of 10⁹, suggesting that thresholds at ≥98% would generate a high percentage of false negatives. In certain examples, a threshold 316 in accordance with the disclosure may be ˜93-95%, 94-95%. 94-96%. 95-96%, or 96-97%.

Consistent with the present disclosure, the distance measure may also be an information-based measure. Distance may also be determined based on Discrete Fourier Transform, (DFT) which may reflect the nucleotide distribution and periodic patterns of a k-mer sequence.

By calibrating validation of CHH1/H2 consensus regions to interval value and k-mer frequency, a maximalized number of validated bp are retained in the resulting diploid assembly. For example, whereas the aggregate of artifacts such as simple indels in a given consensus region may be sufficient to invalidate the entirety of the region, validation at interval value and k-mer frequency may be sufficient to invalidate only those k-mer frequencies within the interval in which such artifacts reside.

In certain examples, (I) can be ≥˜1000 bp, ≥˜2000 bp, ≥˜3000 bp, ≥˜4000 bp, ≥˜5000 bp, ≥˜10,000 bp, ≥˜15,000 bp, or ≥˜20,000 bp. In other examples, (I) can be from ˜1000 bp to 10,000 bp, from ˜1000 bp to 50,000 bp, ˜1000 bp to 100,000 bp, ˜1000 bp to 10⁵ bp, ˜1000 bp to 10⁸ bp, ˜2000 bp to 10,000 bp, from ˜2000 bp to 50,000 bp, ˜2000 bp to 100,000 bp, ˜2000 bp to 10⁵ bp, ˜2000 bp to 108 bp.

In the same or another example, k can be between ˜3 and 10² bp, ˜10 and 10² bp, ˜20 and 10² bp, ˜30 and 10² bp, ˜40 and 10² bp, ˜50 and 10² bp, ˜100 and 10² bp, ˜150 and 10² bp, ˜200 and 10² bp, ˜3 and 10³ bp, ˜10 and 10³ bp, ˜20 and 10³ bp, ˜30 and 10³ bp, ˜40 and 103 bp, ˜50 and 10³ bp, ˜100 and 10³ bp, ˜150 and 10³ bp, ˜200 and 10³, bp ˜3 and 10⁵ bp, ˜10 and 10⁵ bp, ˜20 and 1055 bp, ˜30 and 10⁵ bp, ˜40 and 10⁵ bp, ˜50 and 10⁵ bp, ˜100 and 10⁵ bp, ˜150 and 10⁵ bp, ˜200 and 10⁵ bp.

Validation system 306 may implement a single pass process or a multi-pass process performed at different thresholds and/or conditions. In one example of a multi-pass process, interval sequences deemed invalid in a first pass are run through a second pass at a subinterval. In that regard, subsequences deemed valid in the second pass may be retained, thus maximalizing retention of valid sequences. In the illustrated example of FIG. 3F, a method of a multi-pass validation was performed on both haplotype assemblies of the HG002 benchmark, having a total initial length of consensus regions at 2,631,078,632 bp. In the first pass, at 350, large interval sequences of consensus regions are validated at a 98% similarity, resulting in validated length at 2,569,443,828 bp, 97.54% of initial length. Large interval sequencing failing to pass are broken into TR and NTR contig sets; sequences are extracted by coordinates corresponding to a much smaller subinterval (e.g., TR interval+k for TR, 1000 bp for NTR); and interval sequences in gaps or with inconsistent overlaps are trimmed. In a second pass, at 352 and 354, the TR and NTR sequence interval sets are then separately run through the second pass at 95% similarity, where 258400 bp total length is discarded as <0.95, whereas 61281103 bp are ≥0.95 and are retained to yield a total validated length of consensus regions at 2,630,724,931 bp, 99.98% of the initial length. Thus, a multi-pass process may further maximalizes retention of valid bp.

Referring again to FIG. 3A, the diploid set of validated CHH1-CHH2 consensus region data output from the validation subsystem 306 may be sent directly to the SV caller subsystem 308 or the output may be stored to disk and retrieved from disk by the validation subsystem 306. Validation subsystem 306 may compress the alignment data of the diploid set to a BED file as a diploid assembly for storage and/or downstream processing. In certain examples, validated parental data may also be output for downstream processes, including, e.g., SV call validation processes

The variant caller subsystem 308 may receive the BED file and corresponding CHH1 and CHH2 sequence data compressed to BAM files by first assembly subsystem 302 to perform variant calling on the diploid assembly. The variant caller subsystem 308 may execute an SV calling protocol through operation of an SV caller engine 308a and a call set engine 308b, which may implement hardware and/or software for processing as described herein. For instance, the SV caller engine 308a may implement an SV caller to perform SV calling on the diploid assembly. In certain embodiments SV caller engine 308a operates as an assembly-based SV caller software. Assembly-based SV callers consistent with the present disclosure include, e.g., SVIM-asm, Dipcall, Jasmine, PAV, and Smartie-sv.

In certain examples, the threshold length of SV calling is ≥˜5 bp, ≥˜10 bp, ≥˜15 bp, ≥˜20 bp, ≥˜25 bp, ≥˜30 bp, ≥˜35 bp, ≥˜40 bp, ≥˜45 bp, ≥˜50 bp. Notably, while SVs are commonly considered to be variants of 50 bp or greater, multiple small variants, e.g., deletions are tantamount to SVs in the aggregate. In that regard, protocols utilizing a threshold lower than 50 bp may produce a more comprehensive call set.

In the same or another example, the SV calling protocol includes a combined molecular strategy. For instance, the SV caller engine can execute a multi-sample SV calling incorporating, e.g., one or more of: long-read analysis, paired-end read analysis, split-read analysis, linked read analysis, local reassembly (read-based) analysis, and Strand-seq and Hi-C analysis. In one example, a Hi-C coverage map or matrix may be generated as compliment to a de novo assembly process. A Hi-C sequencing process involves sequencing crosslinked chromatin to obtain information about gDNA sequences distantly spaced in the linear molecule but proximal in 3D space. Hi-C read pairs may thus be used to generate sequence maps that span megabases, making the method useful for mapping read data for detecting large SVs, e.g., translocations.

In addition, the variant caller subsystem 308 may also perform read-based-calling on the CHH1/CHH2 scaffolds compressed to the BAM file by alignment engine 302c. Read-based SV callers consistent with the present disclosure include, e.g., PBSV, Sniffles2, CuteSV, SVIM, and PBHoney. The first assembly subsystem 302 may also implement a read-based workflow in lieu of de novo assembly. To that end, the first assembly subsystem 302 may utilizes the alignment engine 302c to aligns reads, rather than contigs, to a reference genome 312 and the variant caller 306 may, at least in part, perform variant calling using a method appropriate for read-based calling, including methods based on read depth, read pair, and split-read approaches. For example, read depth approaches analyze the alignment depth across the genome for indications of unbalanced CNVs in regions with an elevated read depth caused duplications and regions with a reduced depth caused by deletions. Read pair approaches analyze the relative position and orientation of mapped read pairs. Almost all SV classes can be detected from their characteristic mapping signatures. Read pairs mapping too far apart, for instance, indicate deletions while those mapping too close indicate insertions. Pairs with discordant mapping orientations are indicative of inversions. Split-read (breakpoint) approaches analyze reads that have been split and whose segments have been independently mapped to the reference to produce a better overall alignment. Particularly reads from rearranged regions cannot be mapped linearly to the reference and have to be split up. Similar to read pair approaches, the relative distances between read segments as well as their orientations yield information on virtually all classes of underlying SVs.

In certain examples, the SV caller engine 308a runs pre-filters (or weights) in which block emission of filtered raw calls to lines in the VCF file. Pre-filters may be based, e.g., on confidence or quality scoring, sequencing depth at the call site, SV size, excess heterozygosity, mendelian consistency. Filtered variant calls may be emitted to lines in a VCF (or gVCF) file by the SV caller engine 308a.

In the same or other examples, call set engine 308b may receive the VCF file of reported, filtered variant calls and generate a site record for each call, which may contain information observed at each call site organized by column or field. The fields may include, for each site, #CHROM and POS, for the contig and genomic coordinates at which the variant occurs; ID, for identification of the variant, REF and ALT, for reference and alt alleles; QUAL, for Phred-scaled probability that a REF/ALT polymorphism exists at this site given sequencing data, FILTER, for the names of any post-call filters that the variant fails to pass, or a value [PASS] if the variant passed all filters; and INFO, for annotations of context information represented as tag-value pairs or by ID. The site record for the SV calls may then be output as an SV call set for downstream processing, analysis, and/or application. Filters may reflect, e.g., the proximity of insertion and deletion calls, shadowing of SVs, change of SV call type, the proximity to a gap in the reference assembly, the proximity to the end of a contig, the sufficiency of reads per strand, read gaps within the SV span.

With reference to FIG. 3G, in one example implementation, SV caller engine 308a may operate on SVIM-asm to execute SV calling consistent with the present disclosure. BED file storing diploid assembly may be input and analyzed in three steps. In a first step 320, (COLLECT) SV signatures are collected separately for each CHH1/CHH2-MH1/MH2-PH1/PH2 haplotype set. SVIM-asm extracts six different types of SV signatures from the BED file of validated regions data by analyzing each region one read at a time. Individual alignment segments of each read may be first scanned for intra-alignment discordancies. This type of discordancy may be retrieved from a CIGAR string, a special field in the BED record. The CIGAR string may define the precise read alignment as a string of matches, mismatches, and gaps between the read and the reference. Long gaps in the read or in the reference may represent regions that have been deleted from or inserted into the sequenced genome, respectively, which may be collected as deleted region (DEL) and inserted region (INS) signatures, respectively. Secondly, the alignment segments of each read may be scanned for inter-alignment discordancies, i.e. discordant relative alignment positions and orientations among alignment segments. This type of discordancy may arise when a read is split to enable a better alignment of its segments to the reference. The termination of one alignment segment and the continuation of the alignment at another genomic position may indicate several different SV classes. Therefore, SVIM-asm classifies this type of discordancy in a heuristic fashion to collect the correct type of SV signature.

Initially, the alignment segments of each read may be sorted by their position on the read such that alignment segments involving the first read bases come before segments involving bases later in the read. Then, each pair of adjacent alignment segments, starting with the first and second segment, may be compared. The decision tree may have six possible outcomes. Five of them, representing different SV signature types, may be: (1) deleted segment (DEL)(FIG. 1(a)), (2) inserted region (INS) (FIG. 1(b)), (3) inverted region (INV) (FIG. 1(c)), (4) tandem duplicated region (TAN) (FIG. 1(d)), and (5) translocation breakpoint (BRK) (FIG. 1(f)). The sixth outcome of the tree may capture all remaining cases from which no SV signature is collected.

After completing this heuristic procedure on a given read, a postprocessing step may be performed to collect a sixth SV signature type, interspersed duplicated region (INT) (FIG. 1(e)). Reads covering interspersed duplications may often be represented by split reads whose middle alignment segment has been aligned at a distance from its adjacent segments. Such reads may give rise to two characteristic translocation breakpoints connecting the insertion locus with the start and end of the origin region. Therefore, the translocation breakpoint signatures (BRK) collected for a given read may be scanned for this characteristic pattern and if detected, an interspersed duplicated region signature (INT) may be collected.

In total, the first component of the SVIM-asm pipeline may collect six different types of SV signatures: DEL, INS, INV, TAN, BRK and INT. Some of these evidence types (e.g. INV) indicate one particular SV class (inversion). Others could indicate several possible SV classes. An INS, for instance, can indicate either a duplication or a simple insertion.

In a second step 322 (PAIR), signatures from opposite haplotypes may be compared and paired up if sufficiently similar. Paired signatures from the two haplotypes may be merged into homozygous SV candidates while variants without a partner on the other haplotype may be called as heterozygous SV candidates in a third step 324 (GENOTYPE). Finally, the genotyped SVs may be written to a VCF file.

In the second step 322, similar signatures from opposite haplotypes may be matched. Coarse partitions of signatures in close proximity are formed first. Then, the haplotype sequences for all signatures in a given partition may be generated. The haplotype sequence hap(R, S) of an SV signature S and a reference genome sequence R may be the nucleotide sequence formed by applying the genomic rearrangement defined by S to R. If s=(DEL, chr1, 10100, 20200), for instance, then hap(GRCh37, s) may be the nucleotide sequence that forms when the bases 10100 through 20200 in chromosome 1 may be removed from version 37 of the human reference genome. For every pair x, y 2 S of signatures in a given partition, the edit distance (Levenshtein distance) E(hap(R, x), hap(R, y)) between the haplotype sequences of x and y may be computed. Edit distance may be defined as the minimum number of operations (deletion, insertion, or substitution of one character) required to transform one haplotype sequence into the other.

To compute the edit distance between two signatures, hap(R, x) and hap(R, y) may be generated for the genomic context around x and y and not necessarily for the entire genome. In one implementation, both haplotype sequences may be used for the genomic interval [min(x.start, y.start)−100,max(x.end, y.end)+100] and the library edlib for the computation of the edit distance. To prevent that signatures from the same haplotype are matched, SVIM-asm may enforce a very large distance instead of the actual edit distance between signatures from the same haplotype. Based on the computed distances between their haplotype sequences, the signatures in each partition may be clustered using a hierarchical agglomerative clustering approach. With a low distance threshold for cutting a dendrogram, only very similar signatures (i.e. signatures with similar haplotype sequences) from different haplotypes will be clustered together.

In the third step 324 (GENOTYPE), a genotype estimation of SV signatures may be performed. Clusters of two signatures (from opposite haplotypes) may represent homozygous variants while clusters of only a single signature may represent heterozygous variants. Clusters of more than two signatures will not form due to the large distance enforced between signatures from the same haplotype. In a fourth step 326 (OUTPUT), the six different classes of SV candidates, viz., insertions, inversions, interspersed duplications, tandem duplications, and translocations, are written out in a VCF.

In certain examples, the SV caller engine 308a executes a multiple-caller protocol on the same VCF. [Explain why] For instance, as illustrated in the example system of FIG. 3H, a BED file storing compressed diploid assembly data may be bifurcated to BED files storing respective tandem repeat (TR) regions (repetitive and low complexity regions (RLCR)) and non-tandem repeat regions (NTR) and the bifurcated BED files may be separately analyzed for SVs, including CNV structures. Notably, sequencing-based methods of CNV detection may be difficult in RLCRs. The present system may resolve RLCRs for CNVs using a comprehensive dataset of RLCRs to isolate the TR regions and identify putative CNV structures.

In the example of FIG. 3H, a CHH1H2 diploid assembly compressed to a BED file may first be passed from the validation subsystem 306 to a First TR caller 325 and split by TR/NTR splitter 310 into separate NTR and TR BED files 311a and 311b. The BED files are sent to respective caller engines TR SV caller 308e and SV caller engine 308a. The TR SV caller 308e generates two VCF files for the TR Bed file (phased SV calls in TR and phased SV/CNV calls in TR) and the SV caller engine 308a generates a single VCF file for NTR (Phased SV calls in TR). Putative CNV structures may be deduced and identified as alternative alleles (ALT=‘<CNV:TR>’) in the phased SV calls in TR as follows: (a) copy number (CN)=intervall/|RUS|, (b) Change=CN_Hap−CN_Ref, and (c) if Change<|RUS|/2, InDel Else <CNV:TR>, where RUS refers to the repeat unit sequence of the TR, Hap refers to the TR region sequence, and Ref refers to the reference sequence. By isolating TR segments for SV calling, CNVs responsible for enlargement or contraction of TRs may be deduced and verified with high confidence.

In certain examples, call set engine 308b may receive the VCF file of reported CNV calls and generate a site record for each call generate a site record for putative CNV calls, including records based on application of one or more filters, e.g., CNV overlap with or encapsulation by deletions. In the same or other examples, putative CNV structures may also be evaluated using one or more conformational methods. For example, a deletion breakpoint verification assay may be performed, in which PCR primers are designed to span putative breakpoints and deletion-specific product is sequenced. Read depth uniformity may also confirm putative CNVs. Read-depth uniformity may be measured by the inter-quartile range (IQR), which denote the read depth for which a % of the bases in the reference genome have smaller depths. Different visualization methods may also be used, e.g., BAM confirmation using Integrative Genomics Viewer.

A number of additional processes may be performed in bioinformatics subsystem 204 that are commonly used to ensure quality of results or meet generally accepted best practices for genome assembly, variant calling, or both, including, e.g., Best Practices for Variant Calling with the GATK, e.g., for optical duplicate marking, sorting by coordinate order, base quality score recalibration, genotype refinement, background noise filtering, forced genotyping; NCBI Genome Assembly Model; Genome Reference Consortium rules and guidelines for GRC maintenance of genomes.

FIGS. 4A-B present a flow diagram of an example procedure 450 for generating a CHH1/CHH2 diploid assembly and SV call set using sequence data of CHH1/CHH2-MH1/MH2-PH1/PH2 read sets. The procedure 450 may be performed by one or more portions of a bioinformatics subsystem executed on one or more devices including the bioinformatics subsystem 204 of FIG. 2. As described herein, the one or more portions of the bioinformatics subsystem may be executed by the control circuit of a computing device for performing one or more portions of the procedure 450. For example, one or more control circuits may execute one or more computer-executable instructions or machine-readable instructions stored in memory to perform one or more portions of the procedure 450. In one example embodiment, the one or more portions of the bioinformatics subsystem can include one or more secondary analysis subsystems including one or more of the secondary analysis subsystems illustrated in the example systems of FIGS. 3A and/or 3H. One or more portions of the procedure 450 may be operated locally on a computing device, such as a user device, and/or remotely on another computing device.

Referring to FIG. 4A, the procedure 450 may begin, at 452, by generating three CHH1/CHH2-MH1/MH2-PH1/PH2 read sets. The read sets may be generated by one or more sequencing devices including sequencing device(s) 214 of FIG. 2. The read sets each may be written and compressed to a sequence file (e.g., a FASTA file) for storage and/or downstream processes.

At 454, the stored CHH1/CHH2-MH1/MH2-PH1/PH2 read sets may be phased by the one or more portions of the bioinformatics subsystem according to haplotype into phased CHH1/CHH2-MH1/MH2-PH1/PH2 diploid sets. Phasing the CHH1/CHH2-MH1/MH2-PH1/PH2 read sets may include one or more steps of partitioning CHH1/CHH2 reads using a trio binning technique, as described herein. Example software tools for performing trio binning include HifiAsm, Trio-sga and TrioCano, each of which include modes adapted to analyzes MH1/MH2 and PH1/PH2 short read sets to bin CHH1/CHH2 long reads according to haplotype. Phasing may also include partitioning MH1/MH2 and PH1/PH2 read sets by physical phasing or, alternatively, an orthogonal method, e.g., Hi-C contact map. Example software tools for physical phasing parental data include Beagle (5.2) and ShapeIT, which rely on maximum-likelihood models for phasing; DeepVarient, which uses a classifier model implemented in a convolutional neural network (CNN); or, in example implementations utilizing parental long read data, WhatsHap, using a weighted Minimum Error Correction Problem (wMECP) model.

At 456, one or more portions of the bioinformatics subsystem may include one or more processes for compressing, storing, sending and the phased CHH1/CHH2-MH1/MH2-PH1/PH2 diploid data. For instance, sequence data for each haplotype set of the six phased CHH1/CHH2-MH1/MH2-PH1/PH2 diploid sets may be compressed and written together or individually one or more sequence files (e.g., a bin file) for storage and/or downstream processing.

At 458, the stored phased CHH1/CHH2-MH1/MH2-PH1/PH2 diploid sets may be assembled by one or more portions of the bioinformatics subsystem into phased CHH1/CHH2-MH1/MH2-PH1/PH2 contig sets. In one embodiment, the phased CHH1/CHH2-MH1/MH2-PH1/PH2 read sets may by assembled according to a de novo assembly process. A de novo assembly process may include one or more steps that implement a de Bruijn graph (dBg) or other graph-building model to assemble reads into contigs. For example, a dBg-based model 340 as illustrated in FIG. 3B may be implemented by one or more assembler software tools, in which N1-N4 independently represent a nonspecific nucleobase. A software adapted for short read assembly may be selected as appropriate, e.g., Velvet, ABySS, SOAPdenova, SPAdes, RAMPART. In the same or other examples, a dBg-based model may be implemented in an assembler software adapted for long reads, e.g., HiFiAsm, Canu, Flye, wtdbg2, Shasta. Other graph-based models utilizing an overlap/layout/consensus (OLC) graph or string graph may be used consistent with the present disclosure. OLC-based assemblers include SSAKE, SHARCGS, VCAKE, Celera Assembler, Arachne, and PCAP.

At 460. One or more portions of bioinformatics subsystem may include one or more processes for compressing, storing sequencing data of the phased CHH1/CHH2-MH1/MH2-PH1/PH2 contig sets. For instance, once assembled, the sequencing data of the phased CHH1/CHH2-MH1/MH2-PH1/PH2 contig sets may be compressed together or individually into one or more sequence files (e.g., a FASTA file) and alignment data of the contigs sets may be compressed together or individually into one or more alignment files (e.g., a BAM file) for storage and/or downstream processing.

At 462, the phased CHH1/CHH2-MH1/MH2-PH1/PH2 contig sets may be aligned by one or more portions of the bioinformatics subsystem into phased CHH1/CHH2-MH1/MH2-PH1/PH2 scaffolds. In examples, aligning the phased CHH1/CHH2-MH1/MH2-PH1/PH2 contig sets may include performing one or more steps of generating k-mer seeds from the contig sequence data and looking for matches to a reference genome (e.g., such as reference genome 312 shown in FIG. 3A). The aligning shown at 462 of FIG. 4A may also include one or more steps of indexing the reference genome to a hash table in support of a seed-chain-alignment methodology. For instance, aligning may include (1) indexing k-mer subsequences of a reference genome or genomic region of a reference genome hash table having the hash of a subsequence as its key and the locations of indexed subsequences as its value; (2) querying at coincident k-mer frequency (i.e., seeds) to locate exact matches (or anchors) to the reference; (3) determination of alignment through identification of sets of colinear anchors and scoring the sets based on one or more heuristics.

In examples, one or more steps of aligning may be performed by one or more computational processes for aligning a location of each contig to a location on a reference genome, including a Smith-Waterman (SW) algorithm for local alignment or a global alignment algorithm such as Needleman-Wunsch (NW) algorithm. In examples, one or more steps may be performed by an aligner software, e.g., Minimap2 (mm2), BLAST6, MUMmer7, SSAHA8.

At 464, one or more portions of the bioinformatics subsystem may include one or more processes for compressing, storing, sending, unzipping, and receiving sequence and alignment data of the phased CHH1/CHH2-MH1/MH2-PH1/PH2 scaffolds. For instance, once aligned, the sequencing data of the phased CHH1/CHH2-MH1/MH2-PH1/PH2 scaffolds may be compressed together or individually into one or more alignment files (e.g., a BAM file) for storage and/or downstream processing.

FIG. 4B continues the illustrated procedure 450 of FIG. 4A where, at 466, the phased CHH1/CHH2-MH1/MH2-PH1/PH2 scaffolds are expanded by one or more portions of the bioinformatics subsystem into phased CHH1/CHH2-MH1/MH2-PH1/PH2 regions. In examples, expanding the phased CHH1/CHH2-MH1/MH2-PH1/PH2 scaffolds may include one or more steps of joining adjacent contigs of scaffold into regions according to one or more heuristics, as disclosed herein. In examples, expanding may be performed by computational process that implements a heuristic decision tree, in which, e.g., each heuristic is a predicate expression of alignment between two adjacent contigs.

At 468, one or more portions of bioinformatics subsystem may include one or more processes for compressing, storing, and sending alignment data of the phased CHH1/CHH2-MH1/MH2-PH1/PH2 regions. For instance, alignment data of the phased CHH1/CHH2-MH1/MH2-PH1/PH2 regions may be compressed together or individually into one or more alignment files (e.g., a BED file) for storage and/or downstream processing.

At 470, the phased sets of CHH1/CHH2-MH1/MH2-PH1/PH2 regions may be trimmed by one or more portions of the bioinformatics subsystem into phased sets of CHH1/CHH2-MH1/MH2-PH1/PH2 consensus regions. In examples, trimming the CHH1/CHH2-MH1/MH2-PH1/PH2 regions may include one or more steps of trimming any portions of each region in a given allelic grouping that do not intersect with every other region in the grouping.

At 472, one or more portions of bioinformatics subsystem may include one or more processes for compressing, storing, and sending the alignment data of the phased CHH1/CHH2-MH1/MH2-PH1/PH2 consensus regions. For instance, alignment data of the phased CHH1/CHH2-MH1/MH2-PH1/PH2 consensus regions may be compressed together or individually into one or more alignment files (e.g., a BED file) for storage and/or downstream processing.

At 474, the phased sets of CHH1/CHH2 consensus regions may be validated by one or more portions of the bioinformatics subsystem using composition vector analysis on sequence and alignment data of the phased CHH1/CHH2-MH1/MH2-PH1/PH2 consensus regions. In examples, composition vector analysis on the sequence and alignment data of the phased CHH1/CHH2-MH1/MH2-PH1/PH2 consensus regions may include one or more steps of: (1) calculating k-mer frequency vectors, where the value k can be 1≤k≤(I) and a frequency vector (v) is determined for each (I)−k+1 number (n) of k-strings in a given interval (I) to yield v₁₋ₙ k-mer frequency vectors per interval (I). In one example, (I) can be between ˜10⁵ and 10¹⁰ bp. In the same or another example, k can be between ˜3 and 10² bp; (2) calculating composition vectors of each child consensus region at intervals (I) based on a summing of calculated k-mer frequency vectors; (3) determining a distance between composition vectors of each haplotype child sequence at a given interval and one of the two sequences of respective haplotypes of a transmitting parent (i.e., between M-CHH1-MH1/MH2 and P-CHH2-PH1/PH2, respectively), where distance may be measured as a similarity value between 0 and 1, given by Equation (1), described herein, and (4) determining the validity of a child consensus region at a given interval (I), where validity is established when the similarity value for each sequence of respective haplotypes of the hand one of the two sequences of respective haplotypes of a transmitting parent meets a threshold similarity value.

At 476, one or more portions of bioinformatics subsystem may include one or more processes for compressing, storing, and sending the alignment data of the validated CHH1/CHH2 consensus regions. For instance, alignment data of the validated CHH1/CHH2 consensus regions may be compressed into one or more alignment files (e.g., a BED file) for storage, downstream processing and/or analysis as a diploid assembly.

At 478, SV calling is performed on the CHH1/CHH2 diploid assembly by one or more portions of the bioinformatics subsystem. In examples, variant calling is performed by one or more portions of the bioinformatics subsystem using an SV caller adapted for haplotype-aware SV calling within the validated CHH1/CHH2 consensus regions.

At 480, one or more portions of bioinformatics subsystem may include one or more processes for compressing, storing, and sending SV class and coordinate data of SV calls within the validated CHH1/CHH2 consensus regions. For instance, SV class and coordinate data of the validated CHH1/CHH2 consensus regions may be compressed into a VCF file for storage, downstream processing and/or analysis as an SV call set and/or SV truth set.

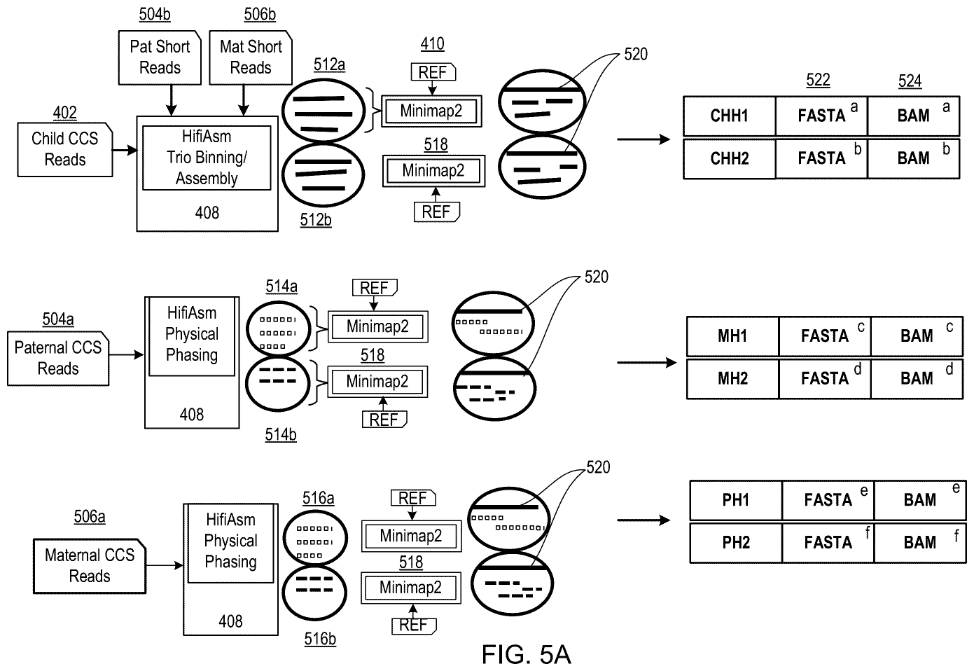

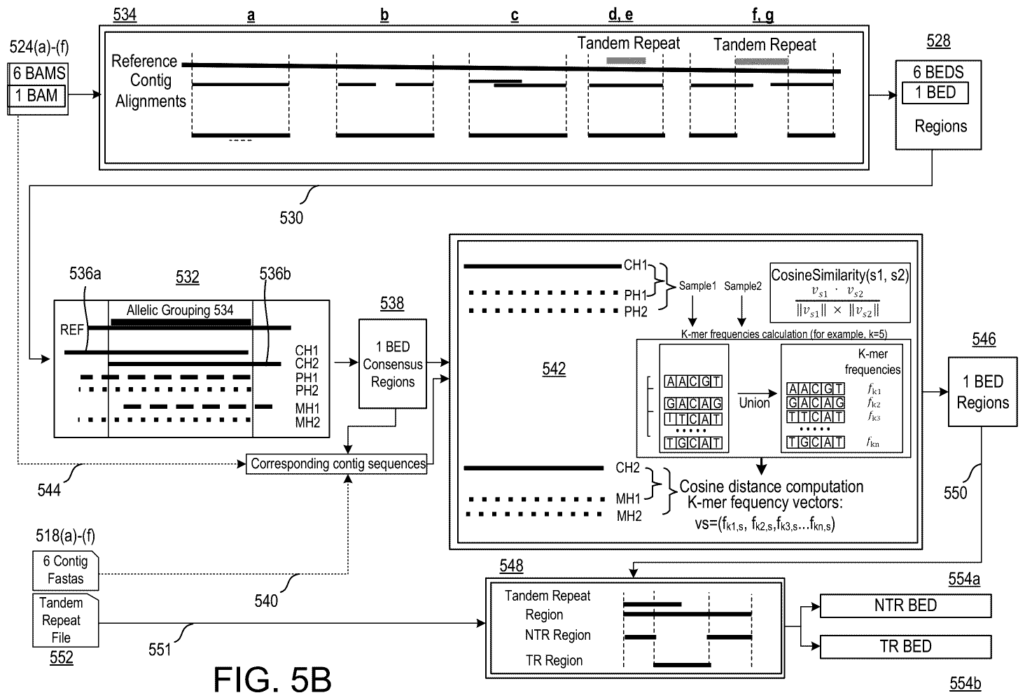

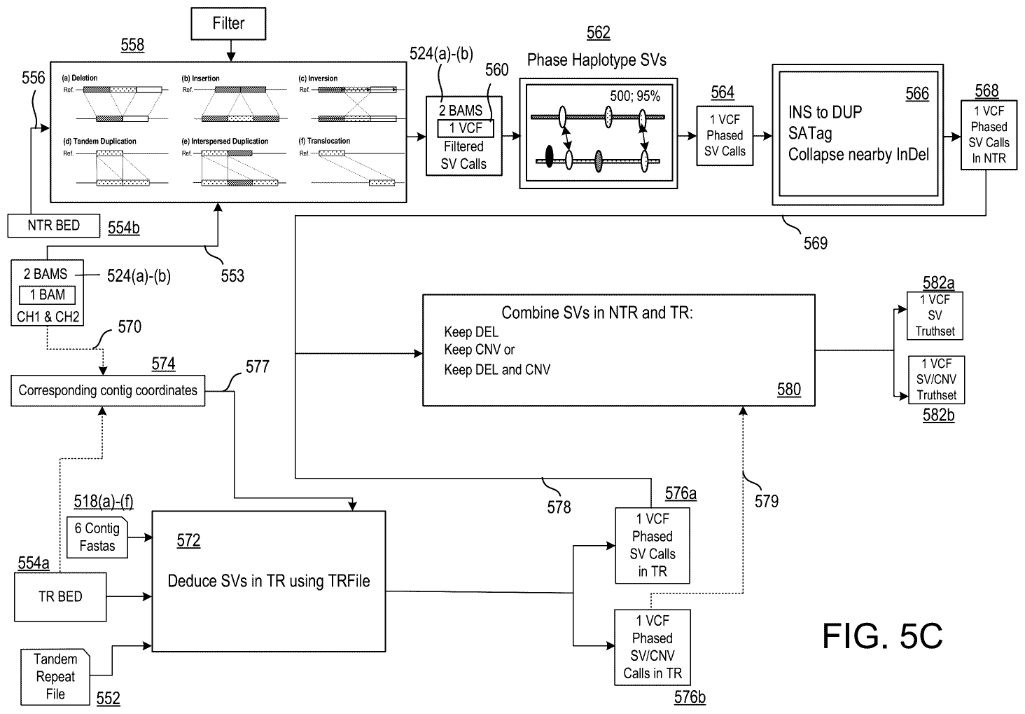

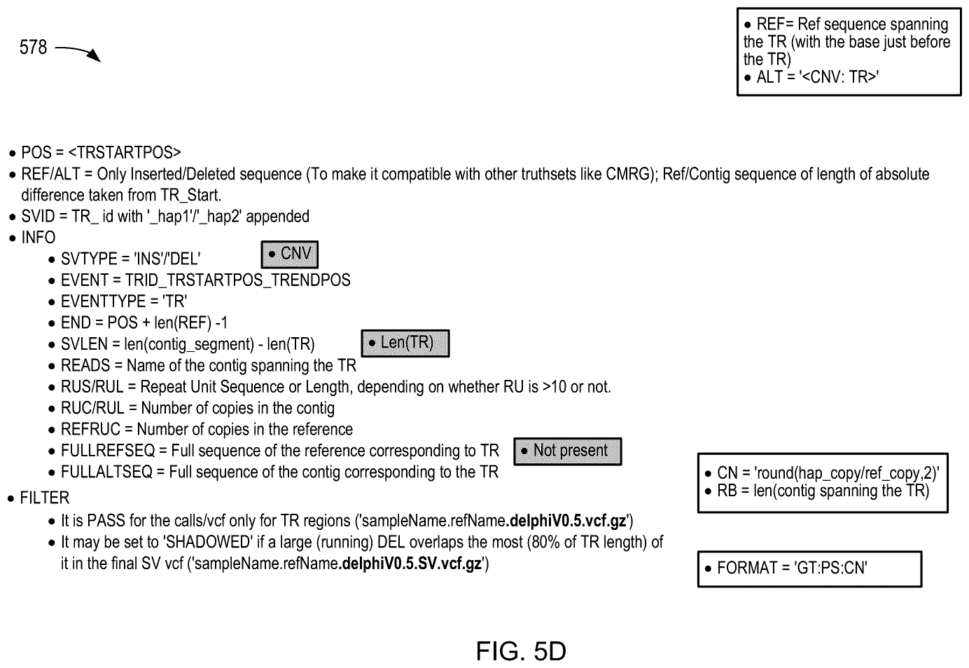

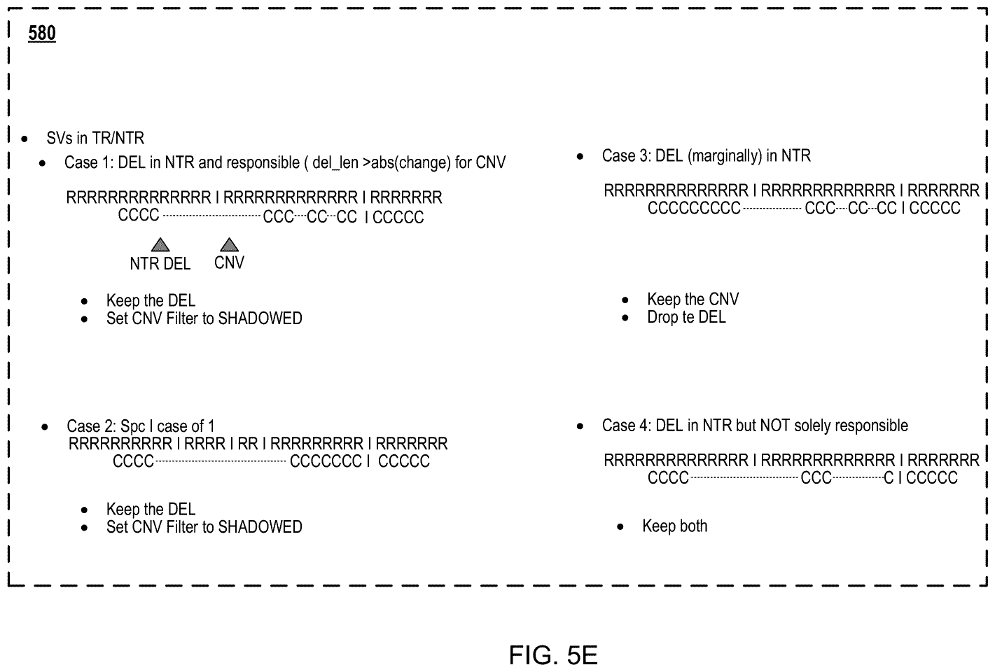

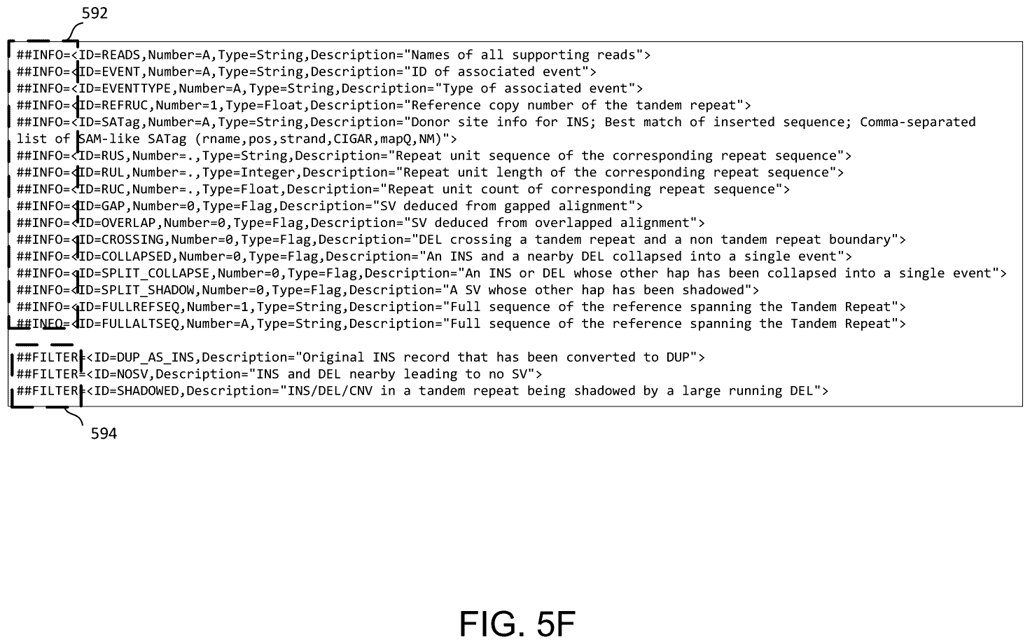

FIGS. 5A-F present example method workflow for generating a CHH1/CHH2 diploid assembly and SV call set using sequence data of CHH1/CHH2-MH1/MH2-PH1/PH2 read sets. The workflow may be performed by one or more portions of a bioinformatics subsystem including the bioinformatics subsystem 204 of FIG. 2. As described herein, the one or more portions of the bioinformatics subsystem may be executed by the control circuit of a computing device for performing one or more portions of the workflow. For example, one or more control circuits may execute one or more computer-executable instructions or machine-readable instructions stored in memory to perform one or more portions of the workflow. In one example embodiment, the one or more portions of the bioinformatics subsystem may include one or more secondary analysis subsystems for implementing one or more portions of the workflow including one or more of the secondary analysis subsystems illustrated in the example system of FIGS. 3A and/or 3H. One or more portions of the workflow may be operated locally on a computing device, such as a user device, and/or remotely on another computing device.

Referring to FIG. 5A, sample sequences input to the workflow may include five CHH1/CHH2-MH1/MH2-PH1/PH2 read sets, viz., child CCS reads 502, Paternal CCS reads 504a, paternal short reads 504b, maternal CCS reads 506b, and maternal short reads 506b. The read sets may be generated by one or more sequencing devices including sequencing device(s) 214 of FIG. 2. CCS reads may be generated by the one or more sequencing devises using a HiFi sequencing technique and reagent chemistry, which provides read depth calibrated to the relatively high random error rates associated with CCS reads. Short reads may be generated by the one or more sequencing devices using a paired-end technique and SBS reagent chemistry. Trio read sets each may be written and compressed to an individual sequence file (e.g., a FASTA file) for input in the workflow.

Referring to FIG. 5A, the input CHH1/CHH2-MH1/MH2-PH1/PH2 read sets 502, 504a, 504b, 506a, and 506b first may be phased by the one or more portions of the bioinformatics subsystem according to haplotype into phased CHH1/CHH2-MH1/MH2-PH1/PH2 diploid sets. Phasing may be performed on the child CCS reads 502a using HifiAsm software 508 executed on one or more processors in a trio binning mode, in which child CCS reads 502a are partitioned into bins 510a and 510b using the input paternal and maternal short reads 504b and 506b. Phasing of paternal and maternal CCS reads 504a and 506b may be performed in parallel using HifiAsm software 508 executed on the same or different processor(s) in a physical phasing mode, in which the CCS reads are partitioned into bins 512a/512b, 514a/514b, and 516a/516b, respectively.

Sequence data for each partitioned haplotype sequence of the phased CHH1/CHH2-MH1/MH2-PH1/PH2 diploid sets may be compressed and written individually in a bin file (not shown) for storage and/or downstream processing.

Once phased, CHH1/CHH2-MH1/MH2-PH1/PH2 diploid sets may be assembled by one or more portions of the bioinformatics subsystem into phased CHH1/CHH2-MH1/MH2-PH1/PH2 contig sets. Assembly may be performed on each haplotype sequence using the HifiAsm software 508 in a de novo assembly mode, in which each haplotype sequence of the diploid sets are assembled into respective contig sets according to a dBg-based graph model. The sequencing data of the phased CHH1/CHH2-MH1/MH2-PH1/PH2 contig sets may be compressed individually to six FASTA files 522(a)-(f) for storage and/or downstream processing.

The phased CHH1/CHH2-MH1/MH2-PH1/PH2 contig sets may then be aligned by one or more portions of the bioinformatics subsystem into phased CHH1/CHH2-MH1/MH2-PH1/PH2 scaffolds. Alignment may be performed on each contig set using a Minimap 2 (mm2) aligner 518 executed on one or more processors, in which each set of contigs is aligned via mapping to a reference genome 520. Resulting alignment data of the phased CHH1/CHH2-MH1/MH2-PH1/PH2 contig sets may be compressed individually to six BAM files 524(a)-(f).

Referring to FIG. 5B, the six BAM 524(a)-(f) may be input to a heuristic model 534 implemented in one or more portions of the bioinformatics subsystem. The heuristic model 534 may expand phased CHH1/CHH2-MH1/MH2-PH1/PH2 scaffolds into larger CHH1/CHH2-MH1/MH2-PH1/PH2 regions by joining adjacent contigs of each respective scaffold according to the following heuristics: (a) continuously adjacent contigs are joined to the same region; (b) with the exception of (d) spaced apart adjacent contigs are joined to the same region; (c) overlapping contigs are joined to the same region; (d) continuously adjacent, spaced-apart, or overlapping contigs in complete sequence alignment with a TR structure are joined to the same region; (e) the contig sequence(s) of (d) in alignment with of a TR structure are retained in the region; (f) spaced apart adjacent contigs flanking and together in partial sequence alignment with a TR structure are joined to separate regions; and (g) the contig sequence(s) of (f) in alignment with of a TR structure are excluded from the separate regions. Resulting alignment data of the phased CHH1/CHH2-MH1/MH2-PH1/PH2 regions may be compressed separately into individual BED files 528.

The individual BED files 528 may be input via path 530 to a trimmer 532 implemented in one or more portions of the bioinformatics subsystem. The trimmer 532 may trim one or more portions of the phased CHH1/CHH2 regions based on orthogonal (allelic) alignment of each corresponding -MH1/MH2-PH1/PH2 region, in which portions of any CHH1/CHH2 region in an allelic grouping 534 not intersecting with each corresponding MH1/MH2-PH1/PH2 region in the grouping (illustrated as portions 536a and 536b) are trimmed from the region. Resulting alignment data of the phased CHH1/CHH2 consensus regions may be compressed into a BED file 538 for storage and/or downstream processing.

The phased sets of CHH1/CHH2 consensus regions may then be validated by one or more portions of the bioinformatics subsystem using composition vector analysis 542 on alignment data of the phased CHH1/CHH2 consensus regions stored to BED file 538, sequence data of corresponding MH1/MH2-PH1/PH2 contig sets stored to FASTA files 518(d)-(f) received via path 540, and alignment data of corresponding MH1/MH2-PH1/PH2 contig sets stored to BAM files 520(c)-(f) received via path 544. Composition vector analysis on the sequence and alignment data of the phased CHH1/CHH2-MH1/MH2-PH1/PH2 consensus regions may include the steps of: (1) calculating k-mer frequency vectors, where the value k can be 1≤k≤(I) and a frequency vector (v) is determined for each (I)−k+1 number (n) of k-strings in a given interval (I) to yield v₁₋ₙ k-mer frequency vectors per interval (I); (2) calculating composition vectors of each child consensus region at intervals (I) based on a summing of calculated k-mer frequency vectors; (3) determining a distance between composition vectors of each haplotype child sequence at a given interval and one of the two sequences of respective haplotypes of a transmitting parent (i.e., between M-CHH1-MH1/MH2 and P-CHH2-PH1/PH2, respectively), where distance may be measured as a similarity value between 0 and 1, given by Equation (1) of FIG. 3C; and (4) determining the validity of a child consensus region at a given interval (I), where validity is established when the similarity value for each sequence of respective haplotypes of the child and one of the two nucleotide sequences of respective haplotypes of a transmitting parent meets a threshold similarity value.

A child consensus region at a given interval (I) may be determined valid if the composition vector of each child sequence of respective haplotypes is within a threshold distance measure of the composition vector of one of the two sequences of respective haplotypes of a transmitting parent (i.e., between M-CHH1-MH1/MH2 and P-CHH2-PH1/PH2, respectively).

A child consensus region at a given interval (I) may be determined valid if the similarity value for each sequence of respective haplotypes of the hand one of the two sequences of respective haplotypes of a transmitting parent meets a threshold 96-97%. Interval (I) sequences of the CHH1/CHH2 consensus regions not meeting this threshold may be discarded. Once validation is completed, alignment data of the validated CHH1/CHH2 consensus regions may be compressed into a BED file 546 for storage, downstream processing, and/or analysis as a diploid assembly.

The alignment data of the validated CHH1/CHH2 consensus regions stored to BED file 546 may then be bifurcated by one or more portions of the bioinformatics subsystem into tandem repeat (TR) and non-tandem repeat (NTR) CHH1/CHH2 consensus regions and the TR and NTR regions are then subject to separate SV calling. Referring to FIG. 5B, the alignment data of the validated CHH1/CHH2 consensus regions stored to BED file 546 is received TR/NTR splitter 548 via path 550. Bifurcation of the validated CHH1/CHH2 consensus regions is performed by the TR/NTR splitter 548 by referencing, via path 551, a TR file 552 listing known TRs and their respective genomic coordinates. Once bifurcated, alignment data of the respective TR and NTR CHH1/CHH2 consensus regions are separately stored to TR BED file 554a and NTR BED file 554b.

Referring to FIG. 5C, the NTR BED file 554b may be sent, via path 556, to SV caller 558 along with BAM files 524(a)-(b) received via path 553. The SV caller 558 may generate a set of filtered calls as to each respective CHH1/CHH2 haplotype using SVIM-asm in a de novo assembly mode, as described herein. SV class and coordinate data of the filtered calls may be compressed into a VCF file 560 and forwarded, along with BAM files 524(a)-(b), to SV phasing process 562 executed in Truvari SV merging software (or BCFtools). In the SV phasing process 562, single SVs called as different events are merged and phased for heterozygosity on both parents. The phased SV calls may then be compressed to a VCF file 564 for storage and/or downstream processing. Reported INS calls in the phased SV calls are scrutinized for DUPs in INS to DUP+SATag process 566. INS of ≥50 bp are first extracted as two sets of sequences: left flank sequence+INS and INS+right flank sequence and mapped to reference genome 520. If a sequence set at a given locus is aligned left flank and right flank at a length≥the INS length, then the reported INS may be marked DUP:TND and the filter for the INS call may be set to INS_AS_DUP. SATag may be used to search the mapped INS calls for transgene terminal sequence(s) that determine integration orientation and genomic position of Ins/dup events. The phased SV calls are written to an NTR VCF file 568 with the INS to DUP metadata.

In parallel, the TR BED file 554a may be received, via path 570, by TR SV caller 572, which may deduce SVs including CNVs in TR structures in the stored TR CHH1/CHH2 consensus regions by referencing known SVs and their respective genomic coordinates stored in TR file 552; corresponding contig coordinates 574 obtained from BAM files 524(a)-(b) received via paths 570 and 577; and FASTA files 518(a)-(f) received via path 571. Putative CNV structures may be deduced in the following string: (a) copy number (CN)=|interval|/|RUS|, (b) Change=CN_Hap−CN_Ref, and (c) if Change<|RUS|/2, InDel Else <CNV:TR>, where RUS refers to the repeat unit sequence of the TR, Hap refers to the TR region sequence, and Ref refers to the reference sequence. Referring to FIG. 5D, putative CNV calls may be reported with POS, REF/ALT, SVID, INFO heads annotated and FILTERs applied as metadata 578.

Referring back to FIG. 5C, the results of SV calling on the TR file may be reported as SV calls written to VCF file 576a and SV/CNV calls written to TR VCF file 576b. TR VCF file 576a, TR VCF file 576b and NTR VCF file 568 are sent via respective paths 578, 579, and 569, to a CNV/DEL process 580, in which reported calls of the TR VCF file 576b and NTR VCF file 568 are recombined to determine whether reported DEL detected in the NTR VCF file 568 may be responsible for a putative CNV reported in TR VCF file 576b in accordance with four cases illustrated in FIG. 5E.

The combined SV calls are written to a VCF SV 4.4 file 582(a) and a VCF SV+CNV file 582(b) for storage and/or analysis as SV truthsets.

FIG. 5F shows a portion of a header of a truthset VCF generated in accordance with the method workflow of FIGS. 5A-C. The header highlights in particular the additional fields appropriate for information for use as a benchmarking tool. The meaning of these additional INFO and FILTER fields has been illustrated in FIGS. 5D and 5E. Broadly, the INFO fields encode the meta information to infer the tandem repeat in which copy number changes have occurred [EVENTTYPE, REFRUC, RUS, RUL, RUC, FULLREFSEQ, FULLALTSEQ], part of the local alignments of the contigs giving rise to SVs in some locus [GAP, OVERLAP, CROSSING, COLLAPSED, SPLIT_COLLAPSE, SPLIT_SHADOW].

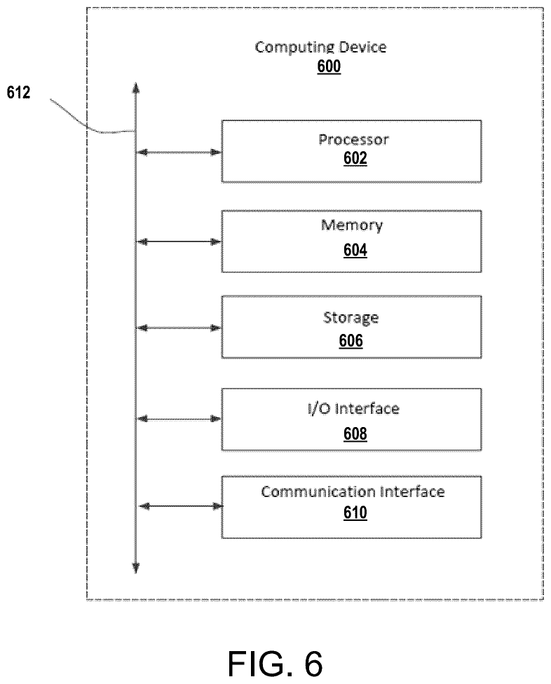

FIG. 6 is a block diagram illustrating an example computing device 600. One or more computing devices such as the computing device 600 may implement one or more features for monitoring and/or changing a version of a sequencing system to service requests from a sequencing application as described herein. For example, the computing device 600 may comprise one or more of the sequencing device(s) 214, the client device(s) 208, and/or the server device(s) 202 shown in FIG. 2. As shown by FIG. 6, the computing device 600 may comprise a processor 602, a memory 604, a storage device 606, an I/O interface 608, and a communication interface 610, which may be communicatively coupled by way of a communication infrastructure 612. The computing device 600 may include fewer or more components than those shown in FIG. 10.

The processor 602 may include hardware for executing instructions, such as those making up a computer application or system. In examples, to execute instructions for operating as described herein, the processor 602 may retrieve (or fetch) the instructions from an internal register, an internal cache, the memory 604, or the storage device 606 and decode and execute the instructions. The memory 604 may be a volatile or non-volatile memory used for storing data, metadata, computer-readable or machine-readable instructions, and/or programs for execution by the processor(s) for operating as described herein. The storage device 606 may include storage, such as a hard disk, flash disk drive, or other digital storage device, for storing data or instructions for performing the methods described herein.

The I/O interface 608 may allow a user to provide input to, receive output from, and/or otherwise transfer data to and receive data from the computing device 600. The I/O interface 608 may include a mouse, a keypad or a keyboard, a touch screen, a camera, an optical scanner, network interface, modem, other known I/O devices or a combination of such I/O interfaces. The I/O interface 608 may include one or more devices for presenting output to a user, including, but not limited to, a graphics engine, a display (e.g., a display screen), one or more output drivers (e.g., display drivers), one or more audio speakers, and one or more audio drivers. The I/O interface 608 may be configured to provide graphical data to a display for presentation to a user. The graphical data may be representative of one or more graphical user interfaces and/or any other graphical content.

The communication interface 610 may include hardware, software, or both. In any event, the communication interface 610 may provide one or more interfaces for communication (such as, for example, packet-based communication) between the computing device 600 and one or more other computing devices and/or networks. The communication may be a wired or wireless communication. As an example, and not by way of limitation, the communication interface 610 may include a network interface controller (NIC) or network adapter for communicating with an Ethernet or other wire-based network or a wireless NIC (WNIC) or wireless adapter for communicating with a wireless network, such as a WI-FI.

Additionally, the communication interface 610 may facilitate communications with various types of wired or wireless networks. The communication interface 610 may also facilitate communications using various communication protocols. The communication infrastructure 612 may also include hardware, software, or both that couples components of the computing device 600 to each other. For example, the communication interface 610 may use one or more networks and/or protocols to enable a plurality of computing devices connected by a particular infrastructure to communicate with each other to perform one or more aspects of the processes described herein. To illustrate, the sequencing process may allow a plurality of devices (e.g., a client device, sequencing device, and server device(s)) to exchange information such as sequencing data and error notifications.

In addition to what has been described herein, the methods and systems may also be implemented in a computer program(s), software, or firmware incorporated in one or more computer-readable media for execution by a computer(s) or processor(s), for example. Examples of computer-readable media include electronic signals (transmitted over wired or wireless connections) and tangible/non-transitory computer-readable storage media. Examples of tangible/non-transitory computer-readable storage media include, but are not limited to, a read only memory (ROM), a random-access memory (RAM), removable disks, and optical media such as CD-ROM disks, and digital versatile disks (DVDs).

Importantly, there is no single adopted computational analysis pipeline used in clinical genetics for SV calling. Rather, there are numerous different or alternative strategies, computational methods, genomic references, and software tools implemented in SV calling workflows in clinical settings, which may vary depending on the particular computational tasks, heuristics, and levels of required sensitivity and specificity for different SV calling objectives, along with resource constraints and the predilections of the particular lab. A number of factors have significant impact on variant calling and thus inform the choices made in implementing a given SV workflow. For example, depending on the objective, e.g., required sensitivity, variant complexity or frequency, the depth and breadth of sequence coverage of the sample may range from gene panels ranging in size from single genes to hundreds of genes, to exome sequences, targeting the approximately 20,000 protein coding genes>100× average depth across the target regions, to whole-genome sequencing, targeting the entire genome with ˜30-60× average sequence depth across the entire genome. Further, a number of computational methods and analytical software tools are tailored to, and thus chosen based on, the particular sequencing device and technique used to generate sample read data, viz., the particular digital read lengths and coverage biases of the various commercial sequencing devices and reagent kits available in the clinical setting. Similarly, a number of computational methods and analytical software tools are tailored to, and thus chosen based on, the sample processing and SV calling approaches, e.g., reference-based read-alignment approaches versus de novo assembly approaches.

The human reference genome (GRCh38) has been the gold standard for mammalian genomes since its first publication in 2001, and there has been considerable investment over the past two decades to increase its accuracy and contiguity. Notwithstanding, even in its current iteration, the number of contiguous sequences (contigs) greatly exceeds the number of chromosomes (1006 contigs versus 24 chromosomes), with most of the major gaps corresponding to large repetitive sequences present in centromeres, acrocentric DNA and segmental duplications.

Moreover, GRCh38 was developed at a time when it was thought that the human genome could be fully represented by a single “golden path” of overlapping sequences from telomere to telomere and that the predominant form of variation understood at the time were single nucleotide polymorphs (SNPs), which could simply be annotated along the path. As a result, 93% of the primary assembly (the assembled chromosomes, unlocalized and unplaced sequences) consists of sequences from a mere eleven genomic clone libraries, and includes only a limited number of alternative contigs (the majority SNPs and InDels) spanning only 60 Mb of the total 2.9 Gb ungapped length of the primary assembly.

Despite tremendous advancement in genetics over the past two decades. updating and diversifying GRCh38 remains a slow, resource-intensive process. GRCh38, for example, was created as a haploid assembly, a mosaic of unlocalized and unplaced sequences of sampled individuals. And, while recent effort has been put in to untangling the reference, it remains a largely haploid assembly. As a result, achieving alignment even of novel sequences to the reference genome may require multiple attempts using multiple alignment methods. And updating the reference at chromosome scale is much more difficult.

In one aspect, end-to-end automated systems implementing an SV calling pipeline for generation of automated assembly of high-quality diploid human reference genomes and SV benchmarks. Because systems and processes of the present disclosure internalize the validation process using trio read inputs, SV calling pipelines may perform diploid assembly on trios within a wide range of genetic and ethnic diversity. Accordingly, SV calling pipelines of the present disclosure may perform not only primary diploid assembly as reference assemblies, but also secondary assemblies useful to refine, diversify and expand existing references through automated compiling of WGS data of multiple sampled trios.

Accordingly, in one aspect, a method is provided for generating a reference assembly according to a system, procedure, or method disclosed herein. In one example, a reference assembly is generated in accordance with the system embodiments of FIG. 3A and/or 3H. In the same or another example, a reference assembly is generated in accordance with the process embodiments of FIGS. 4A-B. In the same or another example, a reference assembly is generated in accordance with the process embodiments of FIGS. 5A-C.

In examples, a method is provided for generating secondary assemblies useful to refine, diversify and expand existing references. In one example, the method yields novel patches for alignment with existing references. Novel patches may comprise alternative loci to an encoding region of the genome. In the same or another example, the method yields fix patched. Fix patches may comprise sequences with corrections to a reference sequence. Fix patches may also comprise improvements to a reference sequence. For example, a fix patch may extend a reference sequence into an existing gap.

In another aspect, a method is provided for generating a benchmark according to a system, procedure, and/or method disclosed herein, wherein the benchmark comprises an SV variant truth set. In one example, a benchmark is generated in accordance with a system embodiment of FIG. 3A and/or 3H. In the same or another example, a reference assembly is generated in accordance with a procedure embodiment of FIGS. 4A-B. In the same or another example, a reference assembly is generated in accordance with a method embodiment of FIGS. 5A-C.

In examples, a method for benchmarking a variant call set is provided. In one example, the method includes performing SV calling in accordance with a system embodiment of FIG. 5A-C, and saving SV calls as an SV truthset in a VCF file. In one example the VCF file may include the metadata reflected in FIG. 5F.

In one example, the method may include calculating, based on the concordance of the variant call set with the SV truthset, one or more of true positive, true negative, false negative, and false positive calls of the variant call set. In the same or another example, the method may include scoring the variant call set to obtain an F1 score based on the calculated true positive and true negative calls as a proportion of total calls of the variant call set. In the same or another example, the method may include scoring the variant call set to obtain a precision score based on calculated true positive calls as a proportion of total calculated true and false positive calls of the variant call set. In the same or another example, the method may include scoring to obtain a recall score based on calculated true positive calls as a proportion of total calculated true positive and false negative calls of the call set. In the same or another example, the method may include assigning, for each call of the variant call set, a pass/fail score based on a minimum threshold score, wherein, each call assigned a pass score is promoted to a reported call (R) and each call assigned a fail score is demoted to a non-call (N). In the same or another example, the method may include scoring the variant calling set based, at least in part, on total R calls as a proportion of total calls.

In another aspect, methods are provided for generated a diploid assembly and variant call set for tertiary analysis, as well as methods of performing tertiary analysis utilizing a diploid assembly and variant call set. In one example, a diploid assembly and variant call set are generated in accordance with a system embodiment of FIG. 3A and/or 3H. In the same or another example, a diploid assembly and variant call set are generated in accordance with a procedure embodiment of FIGS. 4A-B. In the same or another example, a diploid assembly and variant call set are generated in accordance with a method embodiment of FIGS. 5A-C. Tertiary analysis applications may include comprehensive genomic profiling, Genome-wide Association Studies (GWAS), Variant to Function (V2F). QTL mapping, missense studies, loss of function analysis, gain of function analysis, conservation, depletion, deletion analyses.

In one example, a diploid assembly and variant call set for tertiary analysis may be useful to generate a mutation, gene, and transcript annotation in a VCF comprising the variant callset. In the same or another example, a diploid assembly and variant call set for tertiary analysis may be useful to generate a gene expression model in TSV format.

What is claimed is:

1. A system comprising:
at least one processor; and
a non-transitory computer readable medium comprising instructions that, when executed by the at least one processor, cause the system to:
receive a trio dataset of diploid read data of sequenced child and parental genomes;
generate, based on the read data, aligned consensus regions in haplotype-specific (phased) child and parental sets;
validate the child consensus regions at intervals (I), wherein validation for each consensus region is determined based on a distance between composition vectors of corresponding child and transmitting parent sequences at each interval;
output, in an alignment format, a datafile of validated child consensus regions as a diploid assembly;
detect structural variants (SV) within the diploid assembly; and
output, in a variant call format, a datafile of detected SVs as an SV call set.

2. The system of claim 1, wherein composition vector is determined based on a summing of k-mer frequency vectors at a given interval.

3. The system of claim 2, wherein (I) is between ˜10⁵ and 10¹⁰ bp.

4. The system of claim 2, wherein
the value k is 1≤k≤I and the frequency vector (v) is determined for each I−k+1 number (n) of k-strings to yield v1−n k-mer frequency vectors per interval (I).

5. The system of claim 4, wherein k is between ˜3 and 10² bp.

6. The system of claim 4, wherein a child consensus region at a given interval can be determined valid if the composition vector of each child sequence of respective haplotypes is within a threshold distance measure of the composition vector of one of the two sequences of respective haplotypes of a transmitting parent.

7. The system of claim 6, wherein the distance measure is expressed as a similarity value between 0 and 1, given by the formula:
$$similarity = cos ( θ ) = \frac{A \cdot B}{‖ A ‖ ‖ B ‖} = \frac{{\sum }_{i = 1}^{n} {A}_{i} {B}_{i}}{\sqrt{{\sum }_{i = 1}^{n} {A}_{i}^{2}} \sqrt{{\sum }_{i = 1}^{n} {B}_{i}^{2}}} ,$$
wherein
A is the composition vector at a sequence one,
B is the composition vector at a sequence two,
A·B is a dot product of k-mer frequency vectors Av1−n at a sequence one and corresponding k-mer frequency vectors Bv1−n at a sequence two, and
∥A∥ ∥B∥ is the Euclidean norm; and
wherein
sequence one is a haplotype-specific sequence of the child consensus region; and
sequence two is one of the two haplotype-specific sequences of the transmitting parent.

8. The system of claim 8, wherein the threshold is ˜0.93 to 0.97.

9. The system of claim 6, wherein validation is determined in two or more passes,
wherein the instructions, when executed by the at least one processor, cause the system to:
validate, in a first pass, each child consensus region at a first interval (I); and
validate, in a second pass, each region at interval (I) determined to be invalid in the first pass at a second subinterval (Is).

10. The system of claim 1, wherein the SV call set comprises one or more SV classes, wherein each SV class is one or a combination of an insertion, deletion, inversion, duplication, or translocation of >50 bp.

11. The system of claim 10, wherein large segment deletions and duplications of >1000 are classified according to copy number variation (CNV).

12. The method of claim 1, wherein the alignment format datafile comprises a set of genomic coordinates of the diploid assembly.

13. The method of claim 10, wherein the variant call format datafile comprises, for each detected SV, a representation of SV class and location in the diploid assembly.

14. The system of claim 1, wherein the instructions that, when executed by the at least one processor, cause the system to:
output, in respective alignment format datafiles, corresponding tandem repeat (TR) and non-tandem repeat (NTR) datasets of validated child consensus regions;
detect SVs within the NTR dataset;
detect CNV within the TR dataset;
output, in a variant call format, a merged SV call set of detected SVs within the NTR dataset and detected CNV within the TR dataset.

15. The system of claim 1, wherein the instructions that, when executed by the at least one processor, cause the system to:
validate, in a first pass, each child consensus region at interval (I);
generate, from regions determined not to be valid in pass 1, split sets of TR regions and NTR regions; and
validate, in second pass, the split set of TR regions and, separately, the split set of NTR regions at a subinterval (Is).

16. The system of claim 1, wherein the instructions, when executed by the at least one processor, cause the system to:
perform a phenotype study based, at least in part, on one or both of the alignment format and variant call format datafiles.

17. The system of claim 1, wherein the instructions, when executed by the at least one processor, cause the system to:
perform a benchmark analysis of a variant call set based, at least in part, on one or both of the alignment format and variant call format datafiles.

18. The system of claim 1, wherein the instructions, when executed by the at least one processor, cause the system to:
construct or revise a reference genome based, at least in part, on one or both of the alignment format and variant call format datafiles.

19. A method of generating an SV truth set from a trio dataset, the method comprising:
receiving a trio dataset of diploid read data of sequenced child and parental genomes;
generating, based on the read data, aligned consensus regions in haplotype-specific (phased) child and parental sets;
validating the child consensus regions, wherein validation for each consensus region is determined based on composition vectors of corresponding child and transmitting parent sequences at a given interval (I);
outputting, in an alignment format, a datafile of validated child consensus regions as a diploid assembly;
detecting structural variants (SV) within the diploid assembly; and
outputting, in a variant call format, a datafile of detected SVs as an SV call set.

20. The method of claim 19, wherein composition vector is determined based on a summing of k-mer frequency vectors at a given interval.

21. The method of claim 20, wherein (I) is between ˜10⁵ and 10¹⁰ bp.

22. The method of claim 20, wherein
the value k cannot violate 1≤k≤I and the frequency vector (v) is determined for each I−k+1 number (n) of k-strings to yield v₁₋ₙ k-mer frequency vectors per interval (I).

23. The method of claim 22, wherein k is between ˜3 and 10² bp.

24. The method of claim 22, wherein a child consensus region at a given interval can be determined valid if the composition vector of each child sequence of respective haplotypes is within a threshold distance measure of the composition vector of one of the two sequences of respective haplotypes of a transmitting parent.

25. The method of claim 24, wherein the distance measure is expressed as a similarity value between 0 and 1, given by the formula:
$$similarity = cos ( θ ) = \frac{A \cdot B}{‖ A ‖ ‖ B ‖} = \frac{{\sum }_{i = 1}^{n} {A}_{i} {B}_{i}}{\sqrt{{\sum }_{i = 1}^{n} {A}_{i}^{2}} \sqrt{{\sum }_{i = 1}^{n} {B}_{i}^{2}}} ,$$
wherein
A is the composition vector at a sequence one,
B is the composition vector at a sequence two,
A·B is a dot product of k-mer frequency vectors Av₁₋ₙ at a sequence one and corresponding k-mer frequency vectors Bv₁₋ₙ at a sequence two, and
∥A∥ ∥B∥ is the Euclidean norm; and
wherein
sequence one is a haplotype-specific sequence of the child consensus region; and
sequence two is one of the two haplotype-specific sequences of the transmitting parent.

26. The method of claim 25, wherein the threshold is ˜0.93 to 0.97.

27. The method of claim 24, wherein validation further comprises
wherein the instructions, when executed by the at least one processor, cause the system to:
validate, in a first pass, each child consensus region at s first interval (I); and
validate, in a second pass, each region at interval (I) determined to be invalid in the first pass at a second subinterval (Is).

28. The method of claim 19, wherein the SV truth set comprises one or more SV classes, wherein each SV class is one or a combination of an insertion, deletion, inversion, duplication, or translocation of >50 bp.

29. The method of claim 19, wherein large segment deletions and duplications of >1000 are classified according to copy number variation (CNV).

33. The method of claim 29, wherein the alignment format datafile comprises a set of genomic coordinates of the diploid assembly.

31. The method of claim 28, wherein the variant call format datafile comprises, for each detected SV, a representation of SV class and location in the diploid assembly.

32. The method of claim 19 further comprising:
outputting, in respective alignment formats, corresponding tandem repeat (TR) and non-tandem repeat (NTR) datasets of validated child consensus regions;
detecting SVs within the NTR dataset;
detecting CNV within the TR dataset; and
outputting, in a variant call format, a merged SV truth set of detected SVs within the NTR dataset and detected CNV within the TR dataset.

33. The method of claim 19, wherein validating further comprises:
validating, in a first pass, each child consensus region at interval (I);
generating, from regions determined not to be valid in pass 1, split sets of TR regions and NTR regions; and
validating, in second pass, the split set of TR regions and, separately, the split set of NTR regions at a subinterval (Is).

34. The method of claim 19 further comprising:
performing a benchmark analysis of a variant call set based, at least in part, on the SV truth set.

35. The method of claim 19 further comprising:
constructing or revising a reference SV call set, at least in part, on the SV truth set.
